(19) United States
(12) Patent Application Publication
(10) Pub. No.: US 2024/0215926 A1
(43) Pub. Date: Jul. 4, 2024

(54) ADAPTIVE PREDICTIONS BASED ON CONTINUOUS SENSOR MEASUREMENTS

(71) Applicant: UnitedHealth Group Incorporated, Minnetonka, MN (US)

(72) Inventors: An-Ting JHUANG, Chanhassen, MN (US); David Cook, Hopkins, MN (US)

(21) Appl. No.: 18/300,451

(22) Filed: Apr. 14, 2023

Related U.S. Application Data
(60) Provisional application No. 63/477,889, filed on Dec. 30, 2022.

(51) Int. Cl. A61B 5/00; A61B 5/145

(52) U.S. Cl. CPC A61B 5/7275; A61B 5/14532; A61B 5/7264; A61B 5/7282

(57) ABSTRACT

Various embodiments of the present disclosure provide predictive modeling techniques for generating predictive classifications from a plurality of continuous sensor measurements. The techniques may include identifying change points from sensor measurements for an input data object, determining data spikes from the sensor measurements based on the change points, and generating a predictive classification for the input data object based on the data spikes. The predictive classification may correspond to an evaluation time period with one or more sub-time periods. The techniques may include determining a sub-time period classification for each of the sub-time periods of the evaluation time period. The predictive classification may be derived from the sub-time period classifications. Using the predictive classification, an action output may be generated and provided for the input data object.

### CROSS-REFERENCE TO RELATED APPLICATIONS

This application claims the benefit of U.S. Provisional Application No. 63/477,889, entitled “CONTINUOUS GLUCOSE MONITORING BASED TREATMENT SELECTION,” and filed Dec. 30, 2022, the entire contents of which are hereby incorporated by reference.

### BACKGROUND

Various embodiments of the present disclosure address technical challenges related to predictive modeling in complex prediction domains given limitations in existing predictive modeling techniques. Existing processes for generating predictive insights in some prediction domains, such as diabetes diagnostics, are reactive and rely on generic, population-based, sequential, trial and error, protocols. Such techniques may be slow, expensive, and result in predictive insights that are generalized to a population rather than an individual and that fail to adapt to real-time information. Traditionally, these technical shortcomings negate the use of predictive modeling for complex prediction domains, such as type-2 diabetes diagnostics, which are hindered by the complexity of drivers, the burden of self-management, and the inability to provide personalized and precise interventions. Various embodiments of the present disclosure make important contributions to various existing data modeling techniques by addressing these technical challenges.

### BRIEF SUMMARY

Various embodiments of the present disclosure disclose predictive modeling techniques for generating predictive classifications from a plurality of continuous sensor measurements for an input data object. The predictive modeling techniques enable the generation of predictive insights that are tailored and personalized to an input data object at a moment in time. By enabling the efficient interpretation of continuous sensor measurements, some of the predictive modeling techniques described herein may replace existing trial and error protocols with predictive classifications and action outputs that are personalized to an input data object and that are adaptable based on historical observations for the input data object. In this way, some of the predictive modeling techniques of the present disclosure may be used to support (i) the prediction of optimal actions for an input data object without trial and error approaches and (ii) the refinement of the optimal actions over time based on previous action outputs and/or other interventions.

In some embodiments, a computer-implemented method includes identifying, by one or more processors and using a detection model, one or more change points for an input data object, wherein (a) the input data object comprises a plurality of sensor measurements, (b) each sensor measurement of the plurality of sensor measurements was previously generated at a predetermined timing interval over an evaluation time period, and (c) each change point corresponds to a particular sensor measurement of the plurality of sensor measurements; determining, by the one or more processors, at least one data spike from the plurality of sensor measurements based on the one or more change points, wherein the at least one data spike corresponds to a sub-time period of the evaluation time period; determining, by the one or more processors, one or more sub-time period classifications based on the at least one data spike; determining, by the one or more processors, a predictive classification for the input data object based on the one or more sub-time period classifications; and providing, by the one or more processors, an action output for the input data object based on the predictive classification.

In some embodiments, a computing apparatus includes a memory and one or more processors communicatively coupled to the memory. The one or more processors are configured to identify, using a detection model, one or more change points for an input data object, wherein (a) the input data object comprises a plurality of sensor measurements, (b) each sensor measurement of the plurality of sensor measurements was previously generated at a predetermined timing interval over an evaluation time period, and (c) each change point corresponds to a particular sensor measurement of the plurality of sensor measurements; determine at least one data spike from the plurality of sensor measurements based on the one or more change points, wherein the at least one data spike corresponds to a sub-time period of the evaluation time period; determine one or more sub-time period classifications based on the at least one data spike; determine a predictive classification for the input data object based on the one or more sub-time period classifications; and provide an action output for the input data object based on the predictive classification.

In some embodiments, one or more non-transitory computer-readable storage media include instructions that, when executed by one or more processors, cause the one or more processors to identify, using a detection model, one or more change points for an input data object, wherein (a) the input data object comprises a plurality of sensor measurements, (b) each sensor measurement of the plurality of sensor measurements was that are previously generated at a predetermined timing interval over an evaluation time period, and (c) each change point corresponds to a particular sensor measurement of the plurality of sensor measurements; determine at least one data spike from the plurality of sensor measurements based on the one or more change points, wherein the at least one data spike corresponds to a sub-time period of the evaluation time period; determine one or more sub-time period classifications based on the at least one data spike; determine a predictive classification for the input data object based on the one or more sub-time period classifications; and provide an action output for the input data object based on the predictive classification.

### BRIEF DESCRIPTION OF THE DRAWINGS

FIG. 1 illustrates an example computing system in accordance with one or more embodiments of the present disclosure.

FIG. 2 is a schematic diagram showing a system computing architecture in accordance with some embodiments discussed herein.

FIG. 3 is a dataflow diagram showing a first iteration of an iterative predictive modeling technique in accordance with some embodiments discussed herein.

FIG. 4 provides an operational example of a sensor measurement curve in accordance with some embodiments discussed herein.

FIG. 5 is a dataflow diagram showing an iterative predictive modeling technique in accordance with some embodiments discussed herein.

FIG. 6 is a flowchart showing an example of a process for generating predictive insights for a complex prediction domain in accordance with some embodiments discussed herein.

FIG. 7 is a flowchart showing an example of a process for determining a first sub-time period classification for a sub-time period in accordance with some embodiments discussed herein.

FIG. 8 is a flowchart showing an example of a process for determining a second sub-time period classification for a sub-time period in accordance with some embodiments discussed herein.

### DETAILED DESCRIPTION

Various embodiments of the present disclosure are described more fully hereinafter with reference to the accompanying drawings, in which some, but not all embodiments of the present disclosure are shown. Indeed, the present disclosure may be embodied in many different forms and should not be construed as limited to the embodiments set forth herein; rather, these embodiments are provided so that the present disclosure will satisfy applicable legal requirements. The term “or” is used herein in both the alternative and conjunctive sense, unless otherwise indicated. The terms “illustrative” and “example” are used to be examples with no indication of quality level. Terms such as “computing,” “determining,” “generating,” and/or similar words are used herein interchangeably to refer to the creation, modification, or identification of data. Further, “based on,” “based at least in part on,” “based at least on,” “based upon,” and/or similar words are used herein interchangeably in an open-ended manner such that they do not necessarily indicate being based only on or based solely on the referenced element or elements unless so indicated. Like numbers refer to like elements throughout.

### I. COMPUTER PROGRAM PRODUCTS, METHODS, AND COMPUTING ENTITIES

Embodiments of the present disclosure may be implemented in various ways, including as computer program products that comprise articles of manufacture. Such computer program products may include one or more software components including, for example, software objects, methods, data structures, or the like. A software component may be coded in any of a variety of programming languages. An illustrative programming language may be a lower-level programming language such as an assembly language associated with a particular hardware architecture and/or operating system platform. A software component comprising assembly language instructions may require conversion into executable machine code by an assembler prior to execution by the hardware architecture and/or platform. Another example programming language may be a higher-level programming language that may be portable across multiple architectures. A software component comprising higher-level programming language instructions may require conversion to an intermediate representation by an interpreter or a compiler prior to execution.

Other examples of programming languages include, but are not limited to, a macro language, a shell or command language, a job control language, a script language, a database query, or search language, and/or a report writing language. In one or more example embodiments, a software component comprising instructions in one of the foregoing examples of programming languages may be executed directly by an operating system or other software component without having to be first transformed into another form. A software component may be stored as a file or other data storage construct. Software components of a similar type or functionally related may be stored together, such as in a particular directory, folder, or library. Software components may be static (e.g., pre-established, or fixed) or dynamic (e.g., created or modified at the time of execution).

A computer program product may include a non-transitory computer-readable storage medium storing applications, programs, program modules, scripts, source code, program code, object code, byte code, compiled code, interpreted code, machine code, executable instructions, and/or the like (also referred to herein as executable instructions, instructions for execution, computer program products, program code, and/or similar terms used herein interchangeably). Such non-transitory computer-readable storage media include all computer-readable media (including volatile and non-volatile media).

In some embodiments, a non-volatile computer-readable storage medium may include a floppy disk, flexible disk, hard disk, solid-state storage (SSS) (e.g., a solid state drive (SSD), solid state card (SSC), solid state module (SSM), enterprise flash drive, magnetic tape, or any other non-transitory magnetic medium, and/or the like. A non-volatile computer-readable storage medium may also include a punch card, paper tape, optical mark sheet (or any other physical medium with patterns of holes or other optically recognizable indicia), compact disc read only memory (CD-ROM), compact disc-rewritable (CD-RW), digital versatile disc (DVD), Blu-ray disc (BD), any other non-transitory optical medium, and/or the like. Such a non-volatile computer-readable storage medium may also include read-only memory (ROM), programmable read-only memory (PROM), erasable programmable read-only memory (EPROM), electrically erasable programmable read-only memory (EEPROM), flash memory (e.g., Serial, NAND, NOR, and/or the like), multimedia memory cards (MMC), secure digital (SD) memory cards, SmartMedia cards, CompactFlash (CF) cards, Memory Sticks, and/or the like. Further, a non-volatile computer-readable storage medium may also include conductive-bridging random access memory (CBRAM), phase-change random access memory (PRAM), ferroelectric random-access memory (FeRAM), non-volatile random-access memory (NVRAM), magnetoresistive random-access memory (MRAM), resistive random-access memory (RRAM), Silicon-Oxide-Nitride-Oxide-Silicon memory (SONOS), floating junction gate random access memory (FJG RAM), Millipede memory, racetrack memory, and/or the like.

In some embodiments, a volatile computer-readable storage medium may include random access memory (RAM), dynamic random access memory (DRAM), static random access memory (SRAM), fast page mode dynamic random access memory (FPM DRAM), extended data-out dynamic random access memory (EDO DRAM), synchronous dynamic random access memory (SDRAM), double data rate synchronous dynamic random access memory (DDR SDRAM), double data rate type two synchronous dynamic random access memory (DDR2 SDRAM), double data rate type three synchronous dynamic random access memory (DDR3 SDRAM), Rambus dynamic random access memory (RDRAM), Twin Transistor RAM (TTRAM), Thyristor RAM (T-RAM), Zero-capacitor (Z-RAM), Rambus in-line memory module (RIMM), dual in-line memory module (DIMM), single in-line memory module (SIMM), video random access memory (VRAM), cache memory (including various levels), flash memory, register memory, and/or the like. It will be appreciated that where embodiments are described to use a computer-readable storage medium, other types of computer-readable storage media may be substituted for, or used in addition to, the computer-readable storage media described above.

As should be appreciated, various embodiments of the present disclosure may also be implemented as methods, apparatuses, systems, computing devices, computing entities, and/or the like. As such, embodiments of the present disclosure may take the form of an apparatus, system, computing device, computing entity, and/or the like executing instructions stored on a computer-readable storage medium to perform certain steps or operations. Thus, embodiments of the present disclosure may also take the form of an entirely hardware embodiment, an entirely computer program product embodiment, and/or an embodiment that comprises combination of computer program products and hardware performing certain steps or operations.

Embodiments of the present disclosure are described below with reference to block diagrams and flowchart illustrations. Thus, it should be understood that each block of the block diagrams and flowchart illustrations may be implemented in the form of a computer program product, an entirely hardware embodiment, a combination of hardware and computer program products, and/or apparatuses, systems, computing devices, computing entities, and/or the like carrying out instructions, operations, steps, and similar words used interchangeably (e.g., the executable instructions, instructions for execution, program code, and/or the like) on a computer-readable storage medium for execution. For example, retrieval, loading, and execution of code may be performed sequentially such that one instruction is retrieved, loaded, and executed at a time. In some example embodiments, retrieval, loading, and/or execution may be performed in parallel such that multiple instructions are retrieved, loaded, and/or executed together. Thus, such embodiments may produce specifically-configured machines performing the steps or operations specified in the block diagrams and flowchart illustrations. Accordingly, the block diagrams and flowchart illustrations support various combinations of embodiments for performing the specified instructions, operations, or steps.

### II. EXAMPLE FRAMEWORK

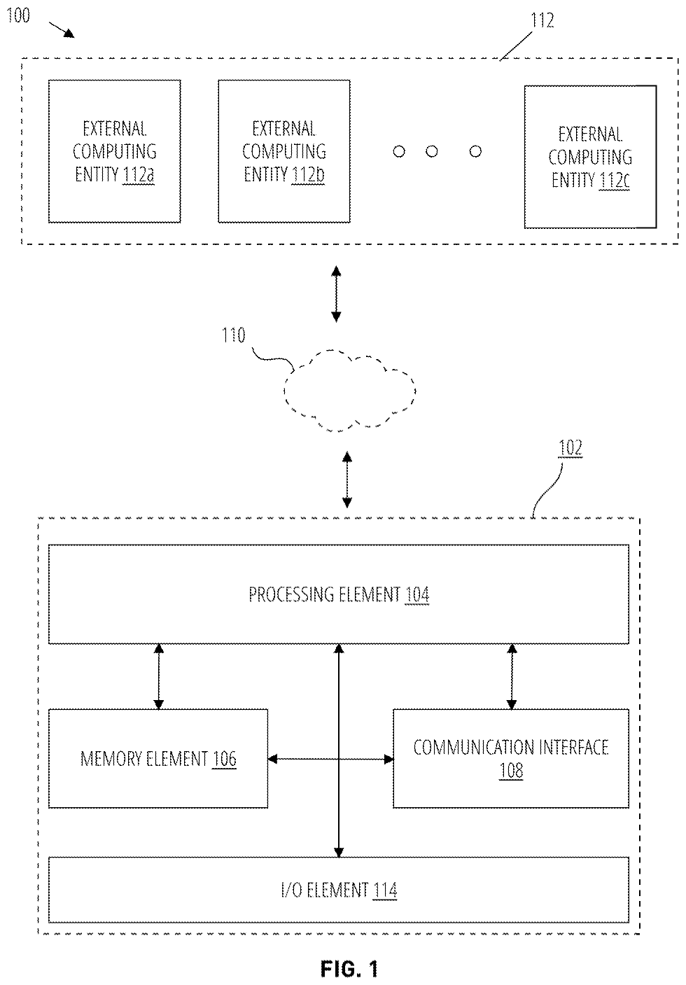

FIG. 1 illustrates an example computing system 100 in accordance with one or more embodiments of the present disclosure. The computing system 100 may include a predictive computing entity 102 and/or one or more external computing entities 112a-c communicatively coupled to the predictive computing entity 102 using one or more wired and/or wireless communication techniques. The predictive computing entity 102 may be specially configured to perform one or more steps/operations of one or more techniques described herein. In some embodiments, the predictive computing entity 102 may include and/or be in association with one or more mobile device(s), desktop computer(s), laptop(s), server(s), cloud computing platform(s), and/or the like. In some example embodiments, the predictive computing entity 102 may be configured to receive and/or transmit one or more datasets, objects, and/or the like from and/or to the external computing entities 112a-c to perform one or more steps/operations of one or more techniques (e.g., predictive modeling techniques, predictive classification techniques, data transformation techniques, and/or the like) described herein.

The external computing entities 112a-c, for example, may include and/or be associated with one or more third-party data sources that may be configured to receive, store, manage, and/or facilitate third-party datasets that may be provided to the predictive computing entity 102. By way of example, the predictive computing entity 102 may include a data processing system that is configured to generate, receive, and/or access sensor measurements from one or more sensing devices (e.g., continuous glucose monitor (CGM) devices), etc.) to determine one or more predictive insights. In some examples, the sensor measurements and/or contextual data associated therewith may be aggregated from one or more of the external computing entities 112a-c to refine one or more predictive insights among other predictive modeling tasks. The external computing entities 112a-c, for example, may be associated with one or more data repositories, cloud platforms, compute nodes, organizations, and/or the like, that may be individually and/or collectively leveraged by the predictive computing entity 102 to obtain data for a prediction domain.

The predictive computing entity 102 may include, or be in communication with, one or more processing elements 104 (also referred to as processors, processing circuitry, digital circuitry, and/or similar terms used herein interchangeably) that communicate with other elements within the predictive computing entity 102 via a bus, for example. As will be understood, the predictive computing entity 102 may be embodied in a number of different ways. The predictive computing entity 102 may be configured for a particular use or configured to execute instructions stored in volatile or non-volatile media or otherwise accessible to the processing element 104. As such, whether configured by hardware or computer program products, or by a combination thereof, the processing element 104 may be capable of performing steps or operations according to embodiments of the present disclosure when configured accordingly.

In one embodiment, the predictive computing entity 102 may further include, or be in communication with, one or more memory elements 106. The memory element 106 may be used to store at least portions of the databases, database instances, database management systems, data, applications, programs, program modules, scripts, source code, object code, byte code, compiled code, interpreted code, machine code, executable instructions, and/or the like being executed by, for example, the processing element 104. Thus, the databases, database instances, database management systems, data, applications, programs, program modules, scripts, source code, object code, byte code, compiled code, interpreted code, machine code, executable instructions, and/or the like, may be used to control certain aspects of the operation of the predictive computing entity 102 with the assistance of the processing element 104.

As indicated, in one embodiment, the predictive computing entity 102 may also include one or more communication interfaces 108 for communicating with various computing entities, e.g., external computing entities 112a-c, such as by communicating data, content, information, and/or similar terms used herein interchangeably that may be transmitted, received, operated on, processed, displayed, stored, and/or the like.

The computing system 100 may include one or more input/output (I/O) element(s) 114 for communicating with one or more users. An I/O element 114, for example, may include one or more user interfaces for providing and/or receiving information from one or more users of the computing system 100. The I/O element 114 may include one or more tactile interfaces (e.g., keypads, touch screens, etc.), one or more audio interfaces (e.g., microphones, speakers, etc.), visual interfaces (e.g., display devices, etc.), and/or the like. The I/O element 114 may be configured to receive user input through one or more of the user interfaces from a user of the computing system 100 and provide data to a user through the user interfaces.

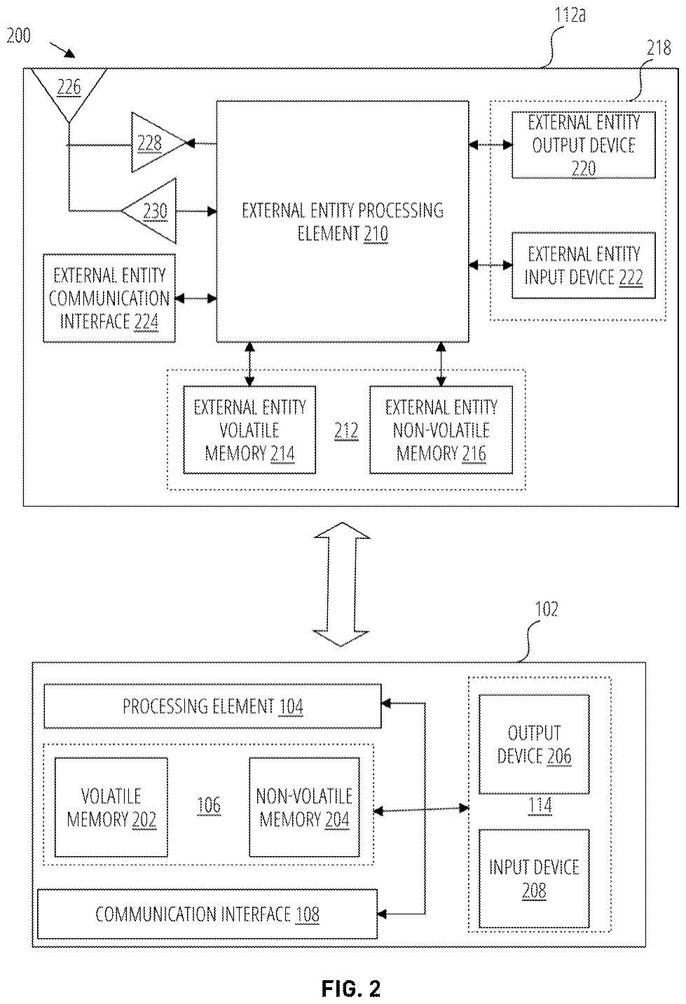

FIG. 2 is a schematic diagram showing a system computing architecture 200 in accordance with some embodiments discussed herein. In some embodiments, the system computing architecture 200 may include the predictive computing entity 102 and/or the external computing entity 112a of the computing system 100. The predictive computing entity 102 and/or the external computing entity 112a may include a computing apparatus, a computing device, and/or any form of computing entity configured to execute instructions stored on a computer-readable storage medium to perform certain steps or operations.

The predictive computing entity 102 may include a processing element 104, a memory element 106, a communication interface 108, and/or one or more I/O elements 114 that communicate within the predictive computing entity 102 via internal communication circuitry, such as a communication bus and/or the like.

The processing element 104 may be embodied as one or more complex programmable logic devices (CPLDs), microprocessors, multi-core processors, coprocessing entities, application-specific instruction-set processors (ASIPs), microcontrollers, and/or controllers. Further, the processing element 104 may be embodied as one or more other processing devices or circuitry including, for example, a processor, one or more processors, various processing devices, and/or the like. The term circuitry may refer to an entirely hardware embodiment or a combination of hardware and computer program products. Thus, the processing element 104 may be embodied as integrated circuits, application specific integrated circuits (ASICs), field programmable gate arrays (FPGAs), programmable logic arrays (PLAs), hardware accelerators, digital circuitry, and/or the like.

The memory element 106 may include volatile memory 202 and/or non-volatile memory 204. The memory element 106, for example, may include volatile memory 202 (also referred to as volatile storage media, memory storage, memory circuitry, and/or similar terms used herein interchangeably). In one embodiment, a volatile memory 202 may include random access memory (RAM), dynamic random access memory (DRAM), static random access memory (SRAM), fast page mode dynamic random access memory (FPM DRAM), extended data-out dynamic random access memory (EDO DRAM), synchronous dynamic random access memory (SDRAM), double data rate synchronous dynamic random access memory (DDR SDRAM), double data rate type two synchronous dynamic random access memory (DDR2 SDRAM), double data rate type three synchronous dynamic random access memory (DDR3 SDRAM), Rambus dynamic random access memory (RDRAM), Twin Transistor RAM (TTRAM), Thyristor RAM (T-RAM), Zero-capacitor (Z-RAM), Rambus in-line memory module (RIMM), dual in-line memory module (DIMM), single in-line memory module (SIMM), video random access memory (VRAM), cache memory (including various levels), flash memory, register memory, and/or the like. It will be appreciated that where embodiments are described to use a computer-readable storage medium, other types of computer-readable storage media may be substituted for, or used in addition to, the computer-readable storage media described above.

The memory element 106 may include non-volatile memory 204 (also referred to as non-volatile storage, memory, memory storage, memory circuitry, and/or similar terms used herein interchangeably). In one embodiment, the non-volatile memory 204 may include one or more non-volatile storage or memory media, including, but not limited to, hard disks, ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like.

In one embodiment, a non-volatile memory 204 may include a floppy disk, flexible disk, hard disk, solid-state storage (SSS) (e.g., a solid-state drive (SSD)), solid state card (SSC), solid state module (SSM), enterprise flash drive, magnetic tape, or any other non-transitory magnetic medium, and/or the like. A non-volatile memory 204 may also include a punch card, paper tape, optical mark sheet (or any other physical medium with patterns of holes or other optically recognizable indicia), compact disc read only memory (CD-ROM), compact disc-rewritable (CD-RW), digital versatile disc (DVD), Blu-ray disc (BD), any other non-transitory optical medium, and/or the like. Such a non-volatile memory 204 may also include read-only memory (ROM), programmable read-only memory (PROM), erasable programmable read-only memory (EPROM), electrically erasable programmable read-only memory (EEPROM), flash memory (e.g., Serial, NAND, NOR, and/or the like), multimedia memory cards (MMC), secure digital (SD) memory cards, SmartMedia cards, CompactFlash (CF) cards, Memory Sticks, and/or the like. Further, a non-volatile computer-readable storage medium may also include conductive-bridging random access memory (CBRAM), phase-change random access memory (PRAM), ferroelectric random-access memory (FeRAM), non-volatile random-access memory (NVRAM), magnetoresistive random-access memory (MRAM), resistive random-access memory (RRAM), Silicon-Oxide-Nitride-Oxide-Silicon memory (SONOS), floating junction gate random access memory (FJG RAM), Millipede memory, racetrack memory, and/or the like.

As will be recognized, the non-volatile memory 204 may store databases, database instances, database management systems, data, applications, programs, program modules, scripts, source code, object code, byte code, compiled code, interpreted code, machine code, executable instructions, and/or the like. The term database, database instance, database management system, and/or similar terms used herein interchangeably may refer to a collection of records or data that is stored in a computer-readable storage medium using one or more database models, such as a hierarchical database model, network model, relational model, entity-relationship model, object model, document model, semantic model, graph model, and/or the like.

The memory element 106 may include a non-transitory computer-readable storage medium for implementing one or more aspects of the present disclosure including as a computer-implemented method configured to perform one or more steps/operations described herein. For example, the non-transitory computer-readable storage medium may include instructions that when executed by a computer (e.g., processing element 104), cause the computer to perform one or more steps/operations of the present disclosure. For instance, the memory element 106 may store instructions that, when executed by the processing element 104, configure the predictive computing entity 102 to perform one or more step/operations described herein.

Embodiments of the present disclosure may be implemented in various ways, including as computer program products that comprise articles of manufacture. Such computer program products may include one or more software components including, for example, software objects, methods, data structures, or the like. A software component may be coded in any of a variety of programming languages. An illustrative programming language may be a lower-level programming language, such as an assembly language associated with a particular hardware framework and/or operating system platform. A software component comprising assembly language instructions may require conversion into executable machine code by an assembler prior to execution by the hardware framework and/or platform. Another example programming language may be a higher-level programming language that may be portable across multiple frameworks. A software component comprising higher-level programming language instructions may require conversion to an intermediate representation by an interpreter or a compiler prior to execution.

Other examples of programming languages include, but are not limited to, a macro language, a shell or command language, a job control language, a script language, a database query, or search language, and/or a report writing language. In one or more example embodiments, a software component comprising instructions in one of the foregoing examples of programming languages may be executed directly by an operating system or other software component without having to be first transformed into another form. A software component may be stored as a file or other data storage construct. Software components of a similar type or functionally related may be stored together, such as in a particular directory, folder, or library. Software components may be static (e.g., pre-established, or fixed) or dynamic (e.g., created or modified at the time of execution).

The predictive computing entity 102 may be embodied by a computer program product include non-transitory computer-readable storage medium storing applications, programs, program modules, scripts, source code, program code, object code, byte code, compiled code, interpreted code, machine code, executable instructions, and/or the like (also referred to herein as executable instructions, instructions for execution, computer program products, program code, and/or similar terms used herein interchangeably). Such non-transitory computer-readable storage media include all computer-readable media such as the volatile memory 202 and/or the non-volatile memory 204.

The predictive computing entity 102 may include one or more I/O elements 114. The I/O elements 114 may include one or more output devices 206 and/or one or more input devices 208 for providing and/or receiving information with a user, respectively. The output devices 206 may include one or more sensory output devices, such as one or more tactile output devices (e.g., vibration devices such as direct current motors, and/or the like), one or more visual output devices (e.g., liquid crystal displays, and/or the like), one or more audio output devices (e.g., speakers, and/or the like), and/or the like. The input devices 208 may include one or more sensory input devices, such as one or more tactile input devices (e.g., touch sensitive displays, push buttons, and/or the like), one or more audio input devices (e.g., microphones, and/or the like), and/or the like.

In addition, or alternatively, the predictive computing entity 102 may communicate, via a communication interface 108, with one or more external computing entities such as the external computing entity 112a. The communication interface 108 may be compatible with one or more wired and/or wireless communication protocols.

For example, such communication may be executed using a wired data transmission protocol, such as fiber distributed data interface (FDDI), digital subscriber line (DSL), Ethernet, asynchronous transfer mode (ATM), frame relay, data over cable service interface specification (DOCSIS), or any other wired transmission protocol. In addition, or alternatively, the predictive computing entity 102 may be configured to communicate via wireless external communication using any of a variety of protocols, such as general packet radio service (GPRS), Universal Mobile Telecommunications System (UMTS), Code Division Multiple Access 2000 (CDMA2000), CDMA2000 1× (1×RTT), Wideband Code Division Multiple Access (WCDMA), Global System for Mobile Communications (GSM), Enhanced Data rates for GSM Evolution (EDGE), Time Division-Synchronous Code Division Multiple Access (TD-SCDMA), Long Term Evolution (LTE), Evolved Universal Terrestrial Radio Access Network (E-UTRAN), Evolution-Data Optimized (EVDO), High Speed Packet Access (HSPA), High-Speed Downlink Packet Access (HSDPA), IEEE 802.9 (Wi-Fi), Wi-Fi Direct, 802.16 (WiMAX), ultra-wideband (UWB), infrared (IR) protocols, near field communication (NFC) protocols, Wibree, Bluetooth protocols, wireless universal serial bus (USB) protocols, and/or any other wireless protocol.

The external computing entity 112a may include an external entity processing element 210, an external entity memory element 212, an external entity communication interface 224, and/or one or more external entity I/O elements 218 that communicate within the external computing entity 112a via internal communication circuitry, such as a communication bus and/or the like.

The external entity processing element 210 may include one or more processing devices, processors, and/or any other device, circuitry, and/or the like described with reference to the processing element 104. The external entity memory element 212 may include one or more memory devices, media, and/or the like described with reference to the memory element 106. The external entity memory element 212, for example, may include at least one external entity volatile memory 214 and/or external entity non-volatile memory 216. The external entity communication interface 224 may include one or more wired and/or wireless communication interfaces as described with reference to communication interface 108.

In some embodiments, the external entity communication interface 224 may be supported by one or more radio circuitry. For instance, the external computing entity 112a may include an antenna 226, a transmitter 228 (e.g., radio), and/or a receiver 230 (e.g., radio).

Signals provided to and received from the transmitter 228 and the receiver 230, correspondingly, may include signaling information/data in accordance with air interface standards of applicable wireless systems. In this regard, the external computing entity 112a may be capable of operating with one or more air interface standards, communication protocols, modulation types, and access types. More particularly, the external computing entity 112a may operate in accordance with any of a number of wireless communication standards and protocols, such as those described above with regard to the predictive computing entity 102.

Via these communication standards and protocols, the external computing entity 112a may communicate with various other entities using means such as Unstructured Supplementary Service Data (USSD), Short Message Service (SMS), Multimedia Messaging Service (MMS), Dual-Tone Multi-Frequency Signaling (DTMF), and/or Subscriber Identity Module Dialer (SIM dialer). The external computing entity 112a may also download changes, add-ons, and updates, for instance, to its firmware, software (e.g., including executable instructions, applications, program modules), operating system, and/or the like.

According to one embodiment, the external computing entity 112a may include location determining embodiments, devices, modules, functionalities, and/or the like. For example, the external computing entity 112a may include outdoor positioning embodiments, such as a location module adapted to acquire, for example, latitude, longitude, altitude, geocode, course, direction, heading, speed, universal time (UTC), date, and/or various other information/data. In one embodiment, the location module may acquire data, such as ephemeris data, by identifying the number of satellites in view and the relative positions of those satellites (e.g., using global positioning systems (GPS)). The satellites may be a variety of different satellites, including Low Earth Orbit (LEO) satellite systems, Department of Defense (DOD) satellite systems, the European Union Galileo positioning systems, the Chinese Compass navigation systems, Indian Regional Navigational satellite systems, and/or the like. This data may be collected using a variety of coordinate systems, such as the Decimal Degrees (DD); Degrees, Minutes, Seconds (DMS); Universal Transverse Mercator (UTM); Universal Polar Stereographic (UPS) coordinate systems; and/or the like. Alternatively, the location information/data may be determined by triangulating a position of the external computing entity 112a in connection with a variety of other systems, including cellular towers, Wi-Fi access points, and/or the like. Similarly, the external computing entity 112a may include indoor positioning embodiments, such as a location module adapted to acquire, for example, latitude, longitude, altitude, geocode, course, direction, heading, speed, time, date, and/or various other information/data. Some of the indoor systems may use various position or location technologies including RFID tags, indoor beacons or transmitters, Wi-Fi access points, cellular towers, nearby computing devices (e.g., smartphones, laptops), and/or the like. For instance, such technologies may include the iBeacons, Gimbal proximity beacons, Bluetooth Low Energy (BLE) transmitters, NFC transmitters, and/or the like. These indoor positioning embodiments may be used in a variety of settings to determine the location of someone or something to within inches or centimeters.

The external entity I/O elements 218 may include one or more external entity output devices 220 and/or one or more external entity input devices 222 that may include one or more sensory devices described herein with reference to the I/O elements 114. In some embodiments, the external entity I/O element 218 may include a user interface (e.g., a display, speaker, and/or the like) and/or a user input interface (e.g., keypad, touch screen, microphone, and/or the like) that may be coupled to the external entity processing element 210.

For example, the user interface may be a user application, browser, and/or similar words used herein interchangeably executing on and/or accessible via the external computing entity 112a to interact with and/or cause the display, announcement, and/or the like of information/data to a user. The user input interface may include any of a number of input devices or interfaces allowing the external computing entity 112a to receive data including, as examples, a keypad (hard or soft), a touch display, voice/speech interfaces, motion interfaces, and/or any other input device. In embodiments including a keypad, the keypad may include (or cause display of) the conventional numeric (0-9) and related keys (#, *, and/or the like), and other keys used for operating the external computing entity 112a and may include a full set of alphabetic keys or set of keys that may be activated to provide a full set of alphanumeric keys. In addition to providing input, the user input interface may be used, for example, to activate or deactivate certain functions, such as screen savers, sleep modes, and/or the like.

### III. EXAMPLES OF CERTAIN TERMS

In some embodiments, the term “input data object” refers to a data entity that describes an initial input to a predictive model. The input data object may include one or more sets of time-based data, such as a plurality of sensor measurements for an entity. For example, the input data object may include a grouping data structure that links one or more sets of time-based data, labels, and/or other characteristics that may be relevant to a predictive classification process. The input data object, for example, may include a data profile for the entity that includes a plurality of sensor measurements over a period of time. In some examples, the input data object may include a unique identifier that is associated with a plurality of sensor measurements for facilitating a predictive classification process.

In some examples, a predictive classification process may include a multi-stage predictive classification process configured to output a predictive classification for the input data object based on the plurality of sensor measurements. The predictive classification process, the predictive classification, and/or the plurality of sensor measurements may be based on the prediction domain. For example, the techniques of the present disclosure may be applied to any of a plurality of different prediction domains, including clinical domains, financial domains, autonomous system domains, and/or the like. In some examples, the prediction domain may include a clinical domain, such as a diabetes diagnostic use case for a patient. In such a case, the input data object may identify a patient and the time-based data may include a plurality of physiological signals for the patient. By way of example, the input data object may include a glucose profile including a plurality of CGM readings for the patient.

In some embodiments, the term “sensor measurement” refers to a data entity that describes a parameter for the input data object. For example, in a clinical domain, a sensor measurement may describe a physiological signal for an entity. In some examples, the sensor measurement may include a time-based CGM measurement for the entity. For instance, the sensor measurement may include a CGM measurement that is generated for the entity at a predetermined time interval. By way of example, the sensor measurement may be generated by a CGM device continuously worn by the entity to provide nearly continuous sensor measurements of the entity's glucose over one or more time periods. For example, using the CGM device, sensor measurements for an entity may be generated at a five-minute frequency. In some examples, the input data object may include a glucose profile for an entity that includes a plurality of time stamped CGM measurements recorded at a five-minute frequency over an evaluation time period.

In some embodiments, the term “evaluation time period” refers to a data entity that describes a time period for generating a predictive classification for an input data object. The evaluation time period may be based on the prediction domain. As one example, for a clinical domain, the evaluation time period may define a period of time for evaluating an entity based on the physiological signals recorded for the entity. By way of example, the evaluation time period may include a fourteen day time window during which a plurality of sensor measurements may be recorded and used to determine a predictive classification for the input data object. The evaluation time period may include any unit of time. In some examples, the evaluation time period may be tailored to a respective input data object and/or a plurality of sensor measurements corresponding to the input data object. By way of example, the evaluation time period may be based on a number of sensor measurements for the input data object, one or more previous predictive classifications for the input data object, and/or the like. In some examples, the evaluation time period may be based on one or more predictive classifications for the input data object. For example, in a clinical domain, the evaluation time period may be based on an efficacy time range for a treatment previously prescribed for an entity.

In some embodiments, the term “sub-time period” refers to a data entity that describes a unit of time within an evaluation time period. The sub-time period may include a portion of the evaluation time period. For example, the evaluation time period may be divided into a plurality of sub-time periods. In some examples, the evaluation time period may be divided evenly to generate a plurality of the sub-time periods of equal duration. In some examples, the evaluation time period may be divided using one or more different criteria to generate a plurality of the sub-time periods of varying durations.

In some examples, the sub-time period may include a unit of time that is proportional to the evaluation time period. A sub-time period, for example, may include a proportion of the evaluation time period. By way of example, in the event that the evaluation time period is fourteen days, the sub-time period may include a twenty-four hour period (e.g., a day, 24-hour time period, etc.) within the fourteen days. As described herein, in some examples, a predictive classification for an input data object may be generated by aggregating sub-time period classifications across each of a plurality of sub-time periods of the evaluation time period.

In some embodiments, the term “change point” refers to a data entity that describes a point of interest for the input data object. A change point, for example, may correspond to a sensor measurement for the input data object. In some examples, a change point may include a sensor measurement that is associated with a change in direction relative to one or more preceding or subsequent sensor measurements for the input data object. By way of example, in a clinical domain, a change point may include a single CGM reading at which there is a sharp change in direction.

In some embodiments, one or more change points are identified from the plurality of sensor measurements using one or more data processing techniques. In some examples, a change point may be identified based on a derivative (e.g., first derivative, etc.) of the plurality of sensor measurements (e.g., after fitting a smooth, continuous curve to discrete data points representative of the sensor measurements, etc.). For example, the change point may be selected from the plurality of sensor measurements in the event the derivative for a sensor measurement exceeds a threshold. In addition, or alternatively, the one or more change points may be identified using a detection model.

In some embodiments, the term “detection model” refers to a data entity that describes parameters, hyper-parameters, and/or defined operations of a rules-based and/or machine learning model (e.g., model including at least one of one or more rule-based layers, one or more layers that depend on trained parameters, coefficients, and/or the like). The detection model may include a statistical and/or machine learning model configured, trained, and/or the like to detect one or more change points from a plurality of sensor measurements. The detection model may include one or more of any type of statistical and/or machine learning model including one or more supervised, unsupervised, semi-supervised, reinforcement learning models, and/or the like. In some examples, the detection model may include multiple models configured to perform one or more different stages of a detection process.

In some examples, the detection model may include a Bayesian ensemble model, such as a Bayesian Estimator of Abrupt change, Season change, and Trend (BEAST) model. The BEAST model, for example, may be configured to process the plurality of sensor measurement to generate a set of time periods during which there is greater than a threshold probability of a change point occurring within the evaluation time period. In some examples, the detection model may output a change point probability for one or more of the plurality of sensor measurements. For example, a plurality of change point probabilities for the plurality of sensor measurements may be output by the Bayesian ensemble model. In some examples, one or more change points may be identified based on a comparison between the plurality of change point probabilities and a sensitivity threshold.

In some embodiments, the term “sensitivity threshold” refers to a data entity that describes a threshold criteria for a change point. The sensitivity threshold, for example, may include a threshold rate of change, a threshold first derivative, a threshold change point probability, and/or the like. In some examples, a sensor measurement may be identified as a change point in the event that the sensor measurement, and/or characteristic (e.g., derivative, change point probability, etc.) thereof, achieves the sensitivity threshold. By way of example, the sensitivity threshold may include a confidence threshold used to tune the detection of change points by the detection model.

In some examples, the sensitivity threshold may be tailored to an input data object. For example, the sensitivity threshold may be increased to reduce the number of change points in the event that an input data object is associated with a plurality of erratic sensor measurements. A plurality of erratic sensor measurements may be defined by one or more high variability metrics. For instance, the input data object may be associated with a plurality of erratic sensor measurements in the event that the sensor measurements for the input data object exceed a threshold data range, threshold variance, threshold standard deviation, and/or the like.

In addition, or alternatively, the sensitivity threshold may be decreased to increase the number of change points in the event that an input data object is associated with a plurality of stable sensor measurements. A plurality of stable sensor measurements may be defined by one or more low variability metrics which, in some examples, may be the converse of the high variability metrics. For instance, the input data object may be associated with a plurality of stable sensor measurements in the event that the sensor measurements are within a threshold data range, threshold variance, threshold standard deviation, and/or the like.

In some embodiments, the term “data spike” refers to a data entity that describes a predictive characteristic of the plurality of sensor measurements. The data spike may include a unit of time within a sub-time period in which one or more or the plurality of sensor measurements express a variability that exceeds a threshold measurement change. For example, the threshold measurement change may define a non-spiking sensor measurement range. A data spike may be detected in the event that at least two sensor measurements within the unit of time exceed the non-spiking sensor range. The at least two sensor measurements, for example, may include a spiking measurement and a start change point. The spiking measurement may include a sensor measurement within the unit of time that exceeds the threshold measurement change relative to the start change point.

By way of example, in a clinical domain a data spike may represent a detected glucose spike for the input data object. In such a case, the data spike may be defined as a unit of time in which a maximum glucose increase over a measurement time period (e.g., two-hour time window, etc.) following a start change point is greater than a threshold measurement change (e.g., sixty mg/dL, etc.). For example, the maximum glucose increase may be defined by the spiking measurement and the start change point. The maximum glucose increase over a measurement time period may satisfy the threshold measurement change in the event that the spiking measurement exceeds the start change point by the threshold measurement change.

In some embodiments, a data spike is defined by a start change point, an endpoint, and a spike duration. The spike duration, for example, may include a unit of time between the start change point and the endpoint.

The start change point may include a first change point of the data spike that is used, with the spiking measurement, to detect the data spike. For example, the start change point may correspond to a start sensor measurement. The data spike may be detected in the event that the difference between the start sensor measurement and the spiking measurement exceeds the threshold measurement change. In some examples, a start change point may be defined as a sensor measurement that is lower than a next subsequent change point by at least a threshold measurement change.

The endpoint may represent a final sensor measurement of the data spike. In some examples, the endpoint may be defined as a sensor measurement (e.g., not necessarily a change point) that is less than or equal to the start sensor measurement. In some examples, for instance in a clinical domain, the plurality of sensor measurements may be analyzed by interpolating between two sensor measurements (e.g., CGM readings, etc.) to surmise a time at which the input data object's sensor measurements (e.g., glucose level, etc.) was exactly equal to the level of the start sensor measurement.

In some embodiments, the term “pre-spike measurement” refers to a data entity that describes a predictive characteristic of the plurality of sensor measurements. The pre-spike measurement may include a sensor measurement that precedes at least one data spike within a sub-time period. In some examples, the pre-spike measurement may include a sensor measurement that precedes a first data spike of a sub-time period by a threshold amount of time, such as thirty minutes, etc.

In some embodiments, the term “evaluation ratio” refers to a data entity that describes a predictive characteristic of the plurality of sensor measurements. The evaluation ratio may be indicative of a data spike time relative to the sub-time period. In some examples, the evaluation ratio may be based on a comparison between one or more data spikes within a sub-time period to one or more non-data spikes within the sub-time period. For example, the plurality of sensor measurements may form a sensor measurement curve including one or more spike areas and one or more non-spike areas. In some examples, the evaluation ratio may be based on one or more areas under the sensor measurement curve. By way of example, the evaluation ratio may be based on an aggregate spike area under the evaluation curve relative to an aggregate non-spike area under the evaluation curve. The aggregate spike area, for example, may include a sum of all spike areas under the evaluation curve. The aggregate non-spike area may include a sum of all non-spike areas under the evaluation curve. In some examples, the evaluation ratio may include the aggregate spike area divided by the aggregate non-spike area.

In some embodiments, the term “threshold pre-spike measurement” refers to a data entity that describes a threshold criteria for a sub-time period classification. The threshold pre-spike measurement may include a threshold sensor measurement. A first sub-time period classification of a first type may be assigned to a sub-time period in the event that the pre-spike measurement satisfies the threshold pre-spike measurement. A second sub-time period classification of the first type may be assigned to the sub-time period in the event that the pre-spike measurement does not satisfy the threshold pre-spike measurement.

The threshold pre-spike measurement may include a static and/or dynamic threshold sensor measurement. In some examples, the threshold pre-spike measurement may include a static measurement for a first predictive classification of an input data object and a dynamic measurement for one or more subsequent predictive classifications.

In some examples, the threshold pre-spike measurement may include a static, pre-defined threshold sensor measurement. The static, pre-defined threshold sensor measurement may be defined by a third-party. By way of example, in a clinical domain, the static, predefined threshold sensor measurement may include a threshold (e.g., 125 mg/dL, etc.) established by a clinical agency, such as the American Diabetes Association Professional Practice Committee.

In some examples, the threshold pre-spike measurement may include a dynamic threshold sensor measurement. The dynamic threshold sensor measurement, for example, may be based on the input data object. For instance, the dynamic threshold sensor measurement may be based on one or more attributes for the input data object, such as one or more demographic attributes, one or more geography-based attributes, one or more activity-based attributes, one or more historical attributes, and/or the like. By way of example, the one or more historical attributes may include one or more previous pre-spike measurements for the input data object and the threshold pre-spike measurement may be based on the one or more previous pre-spike measurements.

In some embodiments, the term “threshold ratio” refers to a data entity that describes a threshold criteria for a sub-time period classification. The threshold ratio may include a threshold evaluation ratio. A first sub-time period classification of a second type may be assigned to a sub-time period in the event that the evaluation ratio satisfies the threshold ratio. A second sub-time period classification of the second type may be assigned to the sub-time period in the event that the evaluation ratio does not satisfy the threshold ratio.

The threshold ratio may include a static and/or dynamic ratio. In some examples, the threshold ratio may include a static ratio for a first predictive classification of an input data object and/or a dynamic ratio for one or more subsequent predictive classifications.

By way of example, the threshold ratio may include a static, pre-defined threshold ratio, such as one, two, and/or the like. In addition, or alternatively, the threshold ratio may include a dynamic ratio that is based on the input data object. For instance, the dynamic ratio may be based on one or more attributes for the input data object, such as one or more demographic attributes, one or more geography-based attributes, one or more activity-based attributes, one or more historical attributes, and/or the like. By way of example, the one or more historical attributes may include one or more previous evaluation ratios for the input data object and the threshold ratio may be based on the one or more previous evaluation ratios.

In some embodiments, the term “sub-time period classification” refers to a data entity that describes a predictive classification for a portion of an evaluation time period. The sub-time period classification may include a predictive classification for a sub-time period based on a subset of the plurality of sensor measurements corresponding to the sub-time period. The sub-time period classification may be based on the prediction domain. In some examples, in a clinical domain, the sub-time period classification may include a phenotype classification, such as fasting phenotype classification and/or a post-prandial phenotype classification. By way of example, a first classification type may include a fasting phenotype classification and/or a second classification type may include a post-prandial phenotype classification.

In some embodiments, the term “first phenotype classification” refers to a data entity that describes a first type of sub-time period classifications for a clinical domain. The first phenotype classification may be indicative of an observable characteristic for an input data object over an evaluation time period. In some examples, in a clinical domain, the first phenotype classification may include a fasting phenotype classification that may be predictive of a predictive classification for the input data object over the evaluation time period. In a clinical domain, the fasting phenotype classification may describe whether an input data object exhibited an elevating fasting glucose for a sub-time period (e.g., a day, 24-hour time period, etc.).

In some embodiments, the term “second phenotype classification” refers to a data entity that describes a second type of sub-time period classifications for a clinical domain. The second phenotype classification may be indicative of an observable characteristic for an input data object over an evaluation time period. In some examples, in a clinical domain, the first phenotype classification may include a postprandial phenotype classification that may be predictive of a predictive classification for the input data object over the evaluation time period. In a clinical domain, the post-prandial phenotype classification may describe whether an input data object exhibited elevated post-prandial glucose for a sub-time period (e.g., a day, 24-hour time period, etc.).

In some embodiments, the term “classification threshold” refers to a data entity that describes a threshold criteria for a predictive classification. The classification threshold may include a threshold number of sub-time period classifications. For example, the classification threshold may include a threshold number of first phenotype classifications (e.g., fasting phenotype classifications, etc.) and/or second phenotype classifications (e.g., post-prandial phenotype classifications, etc.) over an evaluation time period. By way of example, the classification threshold may identify a threshold number of fasting phenotype classifications for determining whether the input data object has an elevated fasting glucose. In addition, or alternatively, the classification threshold may identify a threshold number of post-prandial phenotype classifications over the evaluation time period to determine whether the input data object has an elevated post-prandial glucose phenotype.

In some embodiments, the term “predictive classification” refers to a data entity that describes a predictive output for an input data object. The predictive classification may include one or more predictive categories for the input data object. The one or more predictive categories may be based on the prediction domain. In some examples, in a clinical domain, the predictive classification may include at least one of an elevated fasting glucose only category, an excessive post-prandial glucose elevation only category, and/or a mixed category of elevated fasting glucose and/or excessive post-prandial glucose elevation.

In some embodiments, the term “action output” refers to a data entity that describes a predictive classification-based output for an input data object. The action output may include one or more computing actions, such as issuing one or more alerts or notifications, generating and providing one or more treatment recommendations, generating and rendering one or more instructions, generating and/or providing one or more computing instructions, and/or the like. In some examples, in a clinical domain, the action output may include a clinical plan recommendation based on the predictive classification for the input data object.

### IV. OVERVIEW, TECHNICAL IMPROVEMENTS, AND TECHNICAL ADVANTAGES

Some embodiments of the present disclosure present predictive modeling techniques that improve predictive modeling for a complex prediction domain by generating predictive insights from a plurality of continuous sensor measurements. In some prediction domains, such as clinical prediction domains for diagnosing diabetes, continuous sensor measurements may be unavailable or uninterpretable by traditional predictive modeling techniques. Traditional techniques address this technical shortcoming by using reactive, sequential, trial and error, protocols based on generic, population-based, insights. However, these techniques have several disadvantages including a lack of personalization and adaptability to changes over time. Some embodiments of the present disclosure enable the interpretation of continuous sensor measurements to overcome these disadvantages. To do so, some embodiments of the present disclosure allow for the interpretation of continuous sensor measurements, such as CGM data, to identify predictive classifications that may inform action outputs that are tailored to an individual and adapt to the individual over time. When applied in a diabetes diagnostic setting, these techniques may minimize the number of ineffective treatments prescribed to an individual among other practical benefits.

Some embodiments of the present disclosure provide improvements to predictive classification techniques by enabling the determination of new classifications from continuous sensor measurements. For instance, an evaluation time period may be divided into a plurality of interpretable sub-time periods, each including a set of uninterpretable sensor measurements. Using some of the techniques of the present disclosure, the sensor measurements may be processed by a machine learning model to identify one or more points of interest (e.g., change points) for a respective sub-time period. These points of interest may be adapted to an individual input data object using a dynamic sensitivity threshold. Once identified, the points of interest may be leveraged to identify data spikes within the set of sensor measurements. The timing and the extent of the data spikes may be measured to predict one or more sub-time period classifications for a particular sub-time period. In a clinical setting, the sub-time period classifications may include two dominant clinical subtypes of type-2 diabetes—elevated fasting glucose and excessive post-prandial spiking—that were previously undetectable from continuous sensor measurements.

Some embodiments of the present disclosure provide improvements to predictive modeling techniques by enabling the determination of holistic predictive classifications that are tailored to an individual input data object and adaptable over time. For instance, a predictive classification may be determined for an evaluation time period based on predictive sub-classifications for a plurality of sub-time periods of the evaluation time period. By doing so, a complex, uninterpretable dataset of continuous sensor measurements may be transformed into interpretable classifications reflective of previously undetectable predictive insights from the continuous sensor measurements. These insights may be leveraged to determine a holistic predictive classification that is tailored to an input data object at a particular moment in time. Using these insights, an action output may be generated for an input data object that is specifically tailored to the individual input data object at a particular moment in time. In a clinical setting, the predictive classifications of the present disclosure may empower the selection of treatments based on the dominant type-2 diabetes phenotypes for an individual that may change over time. This ultimately results in effective initial treatments that may be continuously refined based on real-time sensor measurements for the individual.

Example inventive and technologically advantageous embodiments of the present disclosure include (i) predictive classification techniques for transforming uninterpretable sensor measurements to interpretable phenotype classifications, (ii) predictive modeling techniques for generating predictive classifications from the interpretable phenotype classifications, (iii) adaptable tracking and monitoring techniques for refining action outputs for individual input data objects over time, among others.

### V. EXAMPLE SYSTEM OPERATIONS

As indicated, various embodiments of the present disclosure make important technical contributions to data processing and interpretation technology. In particular, systems and methods are disclosed herein that implement predictive modeling techniques for transforming sensor measurements to predictive classifications. Unlike traditional predictive modeling techniques, some of the techniques of the present disclosure leverage continuous sensor measurements and a three-stage approach to deciphering the sensor measurements to determine predictive classifications that are tailored and adaptable to a particular input data object.

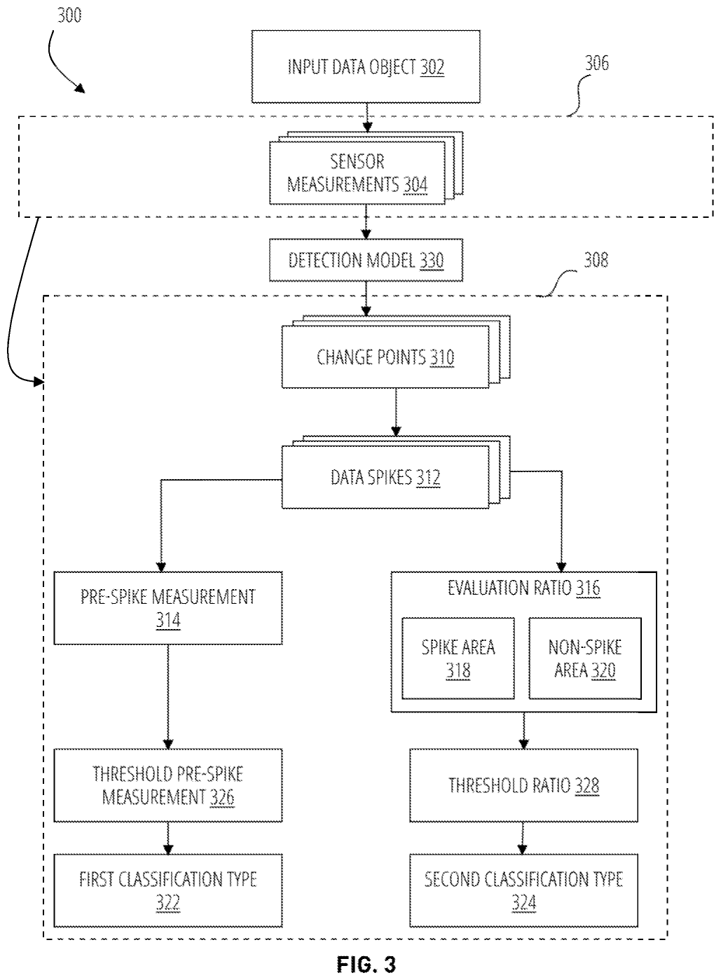

FIG. 3 is a dataflow diagram 300 showing a first iteration of a multi-stage predictive classification process in accordance with some embodiments discussed herein. The dataflow diagram 300 depicts a set of data structures for generating predictive insights for an input data object 302. The predictive insights may include one or more sub-time period classifications, such as the first classification type 322 and/or the second classification type 324. As described herein, these predictive insights may be leveraged in a multi-stage predictive classification process to generate a predictive classification that is specifically tailored to the input data object 302 at a specific moment in time.

In some embodiments, the input data object 302 is a data entity that describes an initial input to a predictive model. The input data object 302 may include one or more sets of time-based data, such as a plurality of sensor measurements 304 for an entity. For example, the input data object 302 may include a grouping data structure that links one or more sets of time-based data, labels, and/or other characteristics that may be relevant to a predictive classification process. The input data object 302, for example, may include a data profile for the entity that includes a plurality of sensor measurements 304 over a period of time. In some examples, the input data object 302 may include a unique identifier that is associated with a plurality of sensor measurements 304 for facilitating a predictive classification process.

In some embodiments, a predictive classification process includes a multi-stage predictive classification process configured to output a predictive classification for the input data object 302 based on the plurality of sensor measurements 304. The predictive classification process, the predictive classification, and/or the plurality of sensor measurements 304 may be based on the prediction domain. For example, the techniques of the present disclosure may be applied to any of a plurality of different prediction domains, including clinical domains, financial domains, autonomous system domains, and/or the like. In some examples, the prediction domain may include a clinical domain, such as a diabetes diagnostic use case for a patient. In such a case, the input data object 302 may identify a patient and the time-based data may include a plurality of physiological signals for the patient. By way of example, the input data object 302 may include a glucose profile including a plurality of CGM measurements for the patient.

In some embodiments, a sensor measurement is a data entity that describes a parameter for the input data object 302. For example, in a clinical domain, a sensor measurement may describe a physiological signal for an entity. In some examples, the sensor measurement may include a time-based CGM measurement for the entity. For instance, the sensor measurement may include a CGM measurement that is generated for the entity at a predetermined time interval. By way of example, the sensor measurement may be generated by a CGM device continuously worn by the entity to provide nearly continuous sensor measurements of the entity's glucose over one or more time periods. For example, using the CGM device, sensor measurements for an entity may be generated at a five-minute frequency. In some examples, the input data object 302 may include a glucose profile for an entity that includes a plurality of time stamped CGM measurements recorded at a five-minute frequency over an evaluation time period 306.

In some embodiments, the evaluation time period 306 is a data entity that describes a time period for generating a predictive classification for an input data object 302. The evaluation time period 306 may be based on the prediction domain. As one example, for a clinical domain, the evaluation time period 306 may define a period of time for evaluating an entity based on the physiological signals recorded for the entity. By way of example, the evaluation time period 306 may include a fourteen day time window during which a plurality of sensor measurements 304 may be recorded and used to determine a predictive classification for the input data object 302. The evaluation time period 306 may include any unit of time. In some examples, the evaluation time period may be tailored to a respective input data object 302 and/or a plurality of sensor measurements 304 corresponding to the input data object 302. By way of example, the evaluation time period 306 may be based on a number of sensor measurements for the input data object 302, one or more previous predictive classifications for the input data object 302, and/or the like. In some examples, the evaluation time period 306 may be based on one or more predictive classifications for the input data object 302. For example, in a clinical domain, the evaluation time period 306 may be based on an efficacy time range for a treatment previously prescribed for an entity.

In some embodiments, the evaluation time period 306 includes a plurality of sub-time periods including the sub-time period 308. For instance, the evaluation time period 306 may be divided into a plurality of portions that may be individually processed to generate predictive insights, such as the first classification type 322 and/or the second classification type 324. By doing so, a plurality of sensor measurements 304 for a complex prediction domain may be divided into a plurality of subunits that, when viewed as whole, may be predictive of a predictive classification for the input data object 302.

In some embodiments, a sub-time period 308 is a data entity that describes a unit of time within the evaluation time period 306. The sub-time period 308 may include a portion of the evaluation time period 306. For example, the evaluation time period 306 may be divided into a plurality of sub-time periods. In some examples, the evaluation time period 306 may be divided evenly to generate a plurality of the sub-time periods of equal duration. In some examples, the evaluation time period 306 may be divided using one or more different criteria to generate a plurality of the sub-time periods of varying durations.

In some embodiments, the sub-time period 308 includes a unit of time that is proportional to the evaluation time period 306. A sub-time period, for example, may include a proportion of the evaluation time period 306. By way of example, in the event that the evaluation time period 306 is fourteen days, the sub-time period may include a twenty-four hour period (e.g., a day, 24-hour time period, etc.) within the fourteen days. As described herein, in some examples, a predictive classification for the input data object 302 may be generated by aggregating sub-time period classifications across each of a plurality of sub-time periods of the evaluation time period 306.

In some embodiments, predictive insights for the input data object 302 are generated based on change points 310 detected from the plurality of sensor measurements 304. For example, one or more change points 310 for the input data object 302 may be identified using a detection model. The input data object 302, for example, may include a plurality of sensor measurements 304 that are previously generated at a predetermined timing interval over the evaluation time period 306. In some examples, each change point may correspond to a particular sensor measurement of the plurality of sensor measurements 304.

In some embodiments, a change point is a data entity that describes a point of interest for the input data object 302. A change point, for example, may correspond to a sensor measurement for the input data object 302. In some examples, a change point may include a sensor measurement that is associated with a change in direction relative to one or more preceding or subsequent sensor measurements for the input data object 302. By way of example, in a clinical domain, a change point may include a single CGM reading at which there is a sharp change in direction.

In some embodiments, the one or more change points 310 are identified from the plurality of sensor measurements using one or more data processing techniques. In some examples, a change point may be identified based on a derivative (e.g., first derivative, etc.) of the plurality of sensor measurements (e.g., after fitting a smooth, continuous curve to discrete data points representative of the sensor measurements, etc.). For example, the change point may be selected from the plurality of sensor measurements 304 in the event the derivative for a sensor measurement exceeds a sensitivity threshold. In addition, or alternatively, the one or more change points may be identified using a detection model 330.

In some embodiments, the detection model 330 is a data entity that describes parameters, hyper-parameters, and/or defined operations of a rules-based and/or machine learning model (e.g., model including at least one of one or more rule-based layers, one or more layers that depend on trained parameters, coefficients, and/or the like). The detection model 330 may include a statistical and/or machine learning model configured, trained, and/or the like to detect the one or more change points 310 from the plurality of sensor measurements 304. The detection model 330 may include one or more of any type of statistical and/or machine learning model including one or more supervised, unsupervised, semi-supervised, reinforcement learning models, and/or the like. In some examples, the detection model 330 may include multiple models configured to perform one or more different stages of a detection process.

In some embodiments, the detection model 330 includes a Bayesian ensemble model, such as a Bayesian Estimator of Abrupt change, Season change, and Trend (BEAST) model. The BEAST model, for example, may be configured to process the plurality of sensor measurements 304 to generate a set of time periods during which there is greater than a threshold probability of a change point occurring within the evaluation time period 306. In some examples, the detection model 330 may output a change point probability for one or more of the plurality of sensor measurements 304. For example, a plurality of change point probabilities for the plurality of sensor measurements 304 may be output by the Bayesian ensemble model. In some examples, the one or more change points 310 may be identified based on a comparison between the plurality of change point probabilities and a sensitivity threshold. For example, a plurality change point probabilities for the plurality of sensor measurements 304 may be generated using the Bayesian ensemble model. The change points 310 may be identified based on a comparison between the plurality of change point probabilities and a sensitivity threshold.

In some embodiments, the sensitivity threshold is a data entity that describes a threshold criteria for a change point. The sensitivity threshold, for example, may include a threshold rate of change, a threshold first derivative, a threshold change point probability, and/or the like. In some examples, a sensor measurement may be identified as a change point in the event that the sensor measurement, and/or characteristic (e.g., derivative, change point probability, etc.) thereof, achieves the sensitivity threshold. By way of example, the sensitivity threshold may include a confidence threshold used to tune the detection of change points by the detection model 330.

In some embodiments, the sensitivity threshold is based on the input data object 302. For example, the sensitivity threshold may be tailored to the input data object 302.

For instance, the sensitivity threshold may be increased to reduce the number of change points in the event that the input data object 302 is associated with a plurality of erratic sensor measurements. A plurality of erratic sensor measurements may be defined by one or more high variability metrics. For instance, the input data object 302 may be associated with a plurality of erratic sensor measurements in the event that the sensor measurements for the input data object 302 exceed a threshold data range, threshold variance, threshold standard deviation, and/or the like.

In addition, or alternatively, the sensitivity threshold may be decreased to increase the number of change points in the event that the input data object 302 is associated with a plurality of stable sensor measurements. A plurality of stable sensor measurements may be defined by one or more low variability metrics which, in some examples, may be the converse of the high variability metrics. For instance, the input data object 302 may be associated with a plurality of stable sensor measurements in the event that the sensor measurements are within a threshold data range, threshold variance, threshold standard deviation, and/or the like.

In some embodiments, a data spike is determined for the input data object 302 based on the change points 310. For instance, the change points 310 may be leveraged to determine one or more data spikes exhibited by the plurality of sensor measurements 304 for the input data object. For example, at least one data spike may be determined from the sensor measurements 304 based on the change points 310. The at least one data spike may correspond to a sub-time period 308 within the evaluation time period 306.

For example, a first data spike may correspond to a first data spike of the sub-time period 308. A Kth data spike may continue to be identified until the end of the sub-time period 308. This can be repeated for each sub-time period of an evaluation time period 306 to determine the plurality of data spikes 312.

In some embodiments, a data spike is a data entity that describes a predictive characteristic of the sensor measurements 304. The data spike may include a unit of time within a sub-time period 308 in which one or more or the plurality of sensor measurements 304 express a variability that exceeds a threshold measurement change. For example, the threshold measurement change may define a non-spiking sensor measurement range. A data spike may be detected in the event that at least two sensor measurements within a measurement time period exceed the non-spiking sensor range. The at least two sensor measurements, for example, may include a spiking measurement and a start change point. The spiking measurement may include a sensor measurement within a measurement time period that exceeds the threshold measurement change relative to the start change point.

In some embodiments, a data spike is determined in response to the identification of at least one spiking measurement. For example, at least one spiking measurement may be identified that corresponds to a particular change point of the change points 310 based on the threshold measurement change. The at least one spiking measurement, for example, may correspond to a sensor measurement within a measurement time period subsequent to the particular change point that exceeds the particular change point by at least the threshold measurement change. The data spike may be determined based on the at least one spiking measurement.

By way of example, in a clinical domain a data spike may represent a detected glucose spike for the input data object 302. In such a case, the data spike may be defined as a unit of time in which a maximum glucose increase over a measurement time period (e.g., two-hour time window, etc.) following a start change point is greater than a threshold measurement change (e.g., sixty mg/dL, etc.). For example, the maximum glucose increase may be defined by the spiking measurement and the start change point. The maximum glucose increase over a measurement time period may satisfy the threshold measurement change in the event that the spiking measurement exceeds the start change point by the threshold measurement change.

In some embodiments, a data spike is defined by a start change point, an endpoint, and a spike duration. The spike duration, for example, may include a unit of time between the start change point and the endpoint.

In some embodiments, the start change point includes a first change point of the data spike that is used, with the spiking measurement, to detect the data spike. For example, the start change point may correspond to a start sensor measurement. The data spike may be detected in the event that the difference between the start sensor measurement and the spiking measurement exceeds the threshold measurement change. In some examples, a start change point may be defined as a sensor measurement that is lower than a next subsequent change point by at least a threshold measurement change.

In some embodiments, the endpoint represents a final sensor measurement of the data spike. In some examples, the endpoint may be defined as a sensor measurement (e.g., not necessarily a change point) that is less than or equal to the start sensor measurement. In some examples, for instance in a clinical domain, the plurality of sensor measurements may be analyzed by interpolating between two sensor measurements (e.g., CGM readings, etc.) to surmise a time at which the input data object's sensor measurements (e.g., glucose level, etc.) was exactly equal to the level of the start sensor measurement.

In some embodiments, one or more sub-time period classifications are determined based on the data spikes 312. For instance, the sub-time period classifications may be determined for the sub-time period 308 based on the data spikes 312 determined from a subset of the sensor measurements 304 corresponding to the sub-time period 308. The sub-time period classifications may include one or more different classifications of different classification types depending on the prediction domain. For example, the sub-time period classifications may include a first classification type 322, a second classification type 324, and/or the like. In a clinical domain, the first classification type 322 may include a first phenotype classification, such as a fasting phenotype classification, and/or the second classification type 324 may include a second phenotype classification, such as a post-prandial phenotype classification.

In some embodiments, the first classification type 322 is determined for the sub-time period 308 based on a pre-spike measurement 314 for the sub-time period 308. For instance, at least one data spike from the data spikes 312 may be determined as the first data spike for the sub-time period 308. In response to determining that the at least one data spike is the first data spike, a pre-spike measurement 314 may be identified for the at least one data spike. The pre-spike measurement 314, for example, may be a sensor measurement of the plurality of sensor measurements 304 that corresponds to a pre-spike measurement time period preceding a start time of the at least one data spike. In some examples, the first classification type 322, such as the fasting phenotype classification, may be determined for the sub-time period 308 based on a comparison between the pre-spike measurement 314 and a threshold pre-spike measurement 326.

In this manner, in some embodiments, a plurality of sensor measurements 304 for a sub-time period 308 is transformed to a first sub-time period classification of a first classification type 322. This can be completed for each sub-time period of the evaluation time period 306 to determine a set of interpretable classifications from a plurality of uninterpretable sensor measurements 304. By way of example, in a clinical domain, the sensor measurements 304 may include a vast amount of CGM readings (e.g., 12*24*14=4,032 glucose readings in 14 days) over a fourteen day time period. Using some of the predictive modeling techniques of the present disclosure, these readings may be transformed to fourteen interpretable classifications, one corresponding to each sub-time period of the evaluation time period 306.

In some embodiments, the pre-spike measurement 314 is a data entity that describes a predictive characteristic of the plurality of sensor measurements 304. The pre-spike measurement 314 may include a sensor measurement that precedes at least one data spike within the sub-time period 308. In some examples, the pre-spike measurement 314 may include a sensor measurement that precedes a first data spike of the sub-time period 308 by a threshold amount of time, such as thirty minutes, etc. The threshold amount of time may be based on the predictive classification, the prediction domain, etc.

In some embodiments, the threshold pre-spike measurement 326 is a data entity that describes a threshold criteria for a sub-time period classification. The threshold pre-spike measurement 326 may include a threshold sensor measurement. The first classification type 322 for the sub-time period 308 may include first value (e.g., indicative of the presence of a fasting phenotype classification) in the event that the pre-spike measurement 314 satisfies the threshold pre-spike measurement 326. The first classification type 322 for the sub-time period 308 may include a second, different value (e.g., indicative of the absence of the fasting phenotype classification) in the event that the pre-spike measurement 314 does not satisfy the threshold pre-spike measurement 326.

In some embodiments, the threshold pre-spike measurement 326 includes a static and/or dynamic threshold sensor measurement. In some examples, the threshold pre-spike measurement 326 may include a static measurement for a first predictive classification of an input data object 302 and a dynamic measurement for one or more subsequent predictive classifications.

In some embodiments, the threshold pre-spike measurement 326 includes a static, pre-defined threshold sensor measurement. The static, pre-defined threshold sensor measurement may be defined by a third-party. By way of example, in a clinical domain, the static, predefined threshold sensor measurement may include a threshold (e.g., 125 mg/dL, etc.) established by a clinical agency, such as the American Diabetes Association Professional Practice Committee.

In some embodiments, the threshold pre-spike measurement 326 includes a dynamic threshold sensor measurement. The dynamic threshold sensor measurement, for example, may be based on the input data object 302. For instance, the dynamic threshold sensor measurement may be based on one or more attributes for the input data object 302, such as one or more demographic attributes, one or more geography-based attributes, one or more activity-based attributes, one or more historical attributes, and/or the like. In some examples, the threshold pre-spike measurement 326 may correspond to a previous evaluation time period for the input data object 302. By way of example, the one or more historical attributes may include one or more previous pre-spike measurements for the input data object 302 that correspond to the one or more previous evaluation time periods. The threshold pre-spike measurement 326 may be based on the one or more previous pre-spike measurements.

In some embodiments, the second classification type 324 is determined for the sub-time period 308 based on an evaluation ratio 316 for the sub-time period 308. For instance, a start change point and an endpoint may be identified for each of the data spikes 312. A spike duration may be determined for each of the data spikes 312 based on their respective start change points and endpoints. An evaluation ratio 316 may be determined based on the spike durations for each of the data spikes 312. For instance, the evaluation ratio 316 may be indicative of a first measurement area corresponding to the data spikes 312 relative to a second measurement area corresponding to one or more portions of the sub-time period 308 that are outside the data spikes 312. In some examples, the second classification type 324, such as the post-prandial phenotype classification, may be determined for the sub-time period 308 based on a comparison between the evaluation ratio 316 and a threshold ratio 328.

In some embodiments, an evaluation ratio 316 is a data entity that describes a predictive characteristic of the plurality of sensor measurements 304. The evaluation ratio 316 may be indicative of a data spike time relative to the sub-time period 308. In some examples, the evaluation ratio 316 may be based on a comparison between one or more data spikes within a sub-time period 308 to one or more non-data spikes within the sub-time period 308. For example, the plurality of sensor measurements 304 may form a sensor measurement curve including one or more spike areas 318 and one or more non-spike areas 320. In some examples, the evaluation ratio 316 may be based on one or more areas under the measurement curve.

By way of example, the evaluation ratio 316 may be based on an aggregate spike area under the evaluation curve relative to an aggregate non-spike area under the evaluation curve. The aggregate spike area, for example, may include a sum of all spike areas 318 under the evaluation curve. The aggregate non-spike area may include a sum of all non-spike areas 320 under the evaluation curve. In some examples, the aggregate spike area may include an area under the sensor measurement curve that is above the pre-spike measurement 314 for the sub-time period 308. In addition, or alternatively, the aggregate non-spike areas may include an area under the sensor measurement curve that is below the pre-spike measurement 314 for the sub-time period 308. In some examples, the evaluation ratio 316 may include the aggregate spike area divided by the aggregate non-spike area.

By way of example, in a clinical domain, the sensor measurement curve may include a CGM curve.

The aggregate spike area may include the sum of areas under the CGM curve for spikes in a sub-time period 308, such as a day, a 24-hour time period, and/or the like. In some examples, the pre-spike measurement 314 may include a fasting glucose for a day. The aggregate spike area may include the sum of areas that are under the CGM curve and above the identified fasting glucose for a given day across all data spikes 312. This sum may represent the data spikes 312 contribution to glucose. The aggregate non-spike area may include the sum of areas under the CGM curve for non-spikes in a sub-time period 308, such as a day, a 24-hour time period, and/or the like. The aggregate non-spike area may include the sum of areas under the CGM curve and above a normal threshold across non-spikes. The threshold should reflect normal (e.g., non-diabetic) fasting glucose levels, such as 100 mg/dL.

In some embodiments, a threshold ratio 328 is a data entity that describes a threshold criteria for a sub-time period classification. The threshold ratio 328 may include a threshold evaluation ratio.

The second classification type 324 for the sub-time period 308 may include a first value (e.g., indicative of the presence of a post-prandial phenotype) in the event that the evaluation ratio 316 satisfies the threshold ratio 328 and/or a second, different value (e.g., indicative of an absence of the post-prandial phenotype) in the event that the evaluation ratio 316 does not satisfy the threshold ratio 328.

The threshold ratio may include a static and/or dynamic ratio. In some examples, the threshold ratio may include a static ratio for a first predictive classification of the input data object 302 and/or a dynamic ratio for one or more subsequent predictive classifications.

By way of example, the threshold ratio 328 may include a static, pre-defined threshold ratio, such as one, two, and/or the like. In addition, or alternatively, the threshold ratio 328 may include a dynamic ratio that is based on the input data object 302. For instance, the dynamic ratio may be based on one or more attributes for the input data object 302, such as one or more demographic attributes, one or more geography-based attributes, one or more activity-based attributes, one or more historical attributes, and/or the like. In some examples, the threshold ratio 328 may correspond to a previous evaluation time period for the input data object 302. By way of example, the one or more historical attributes may include one or more previous evaluation ratios for the input data object 302 that correspond to the one or more previous evaluation time periods. The threshold ratio 328 may be based on the one or more previous threshold ratios.

In some embodiments, a sub-time period classification is a data entity that describes a predictive classification for a portion of an evaluation time period 306. The sub-time period classification may include a predictive classification for a sub-time period 308 based on a subset of the plurality of sensor measurements 304 corresponding to the sub-time period 308. The sub-time period classification may be based on the prediction domain. In some examples, in a clinical domain, the sub-time period classification may include a phenotype classification, such as fasting phenotype classification and/or a post-prandial phenotype classification. By way of example, a first classification type 322 may include a first phenotype classification, such as a fasting phenotype classification and/or a second classification type 324 may include a second phenotype classification, such as a post-prandial phenotype classification.

In some embodiments, the first phenotype classification is a data entity that describes a first type of sub-time period classifications for a clinical domain. The first phenotype classification may be indicative of an observable characteristic for an input data object 302 over an evaluation time period 306. In some examples, in a clinical domain, the first phenotype classification may include a fasting phenotype classification that may be predictive of a predictive classification for the input data object 302 over the evaluation time period. In a clinical domain, the fasting phenotype classification may describe whether an input data object 302 exhibited an elevating fasting glucose for a sub-time period 308 (e.g., a day, 24-hour time period, etc.).

In some embodiments, the second phenotype classification is a data entity that describes a second type of sub-time period classifications for a clinical domain. The second phenotype classification may be indicative of an observable characteristic for an input data object 302 over an evaluation time period 306. In some examples, in a clinical domain, the first phenotype classification may include a postprandial phenotype classification that may be predictive of a predictive classification for the input data object 302 over the evaluation time period 306. In a clinical domain, the post-prandial phenotype classification may describe whether an input data object 302 exhibited elevated post-prandial glucose for a sub-time period 308 (e.g., a day, 24-hour time period, etc.).

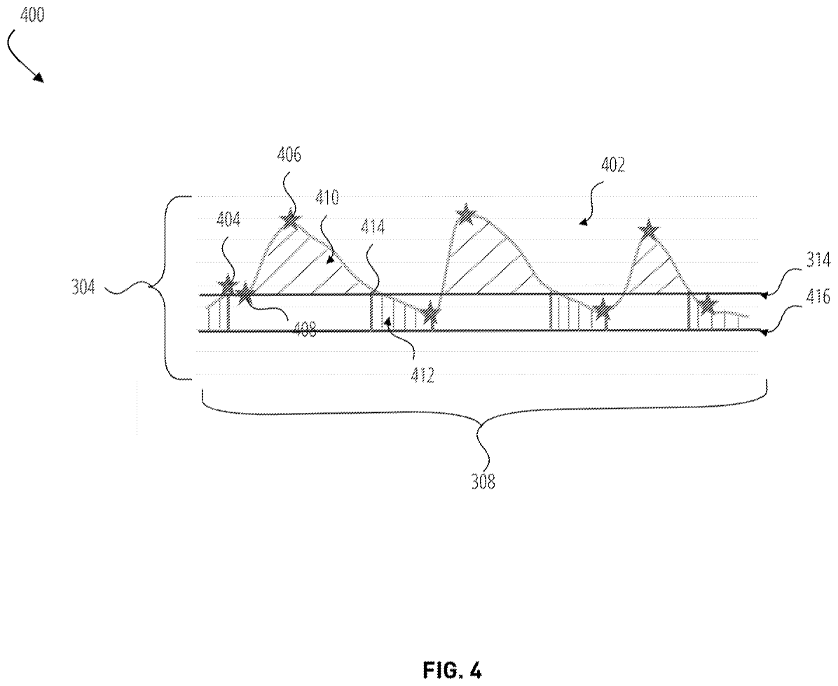

As described herein, the sub-time period classifications may be determined for an input data object 302 based on a plurality of sensor measurements over the sub-time period 308. In some examples, the sensor measurements over the sub-time period 308 may be represented as a sensor measurement curve. An example of the sensor measurement curve will now further be described with reference to FIG. 4.

FIG. 4 provides an operational example 400 of a sensor measurement curve 402 in accordance with some embodiments discussed herein. The sensor measurement curve 402 may include a plurality of time-stamped sensor measurements 304 within a sub-time period 308. The sensor measurements 304 may be sequentially plotted based on their respective time stamps to generate the sensor measurement curve 402.

The sensor measurement curve 402 includes a plurality of change points, such as change point 404. In addition, the sensor measurement curve 402 includes a plurality of data spikes that respectively define spiking areas, such the spiking area 410. The spiking area 410, for example, may be defined by a data spike with a start change point 408 and an endpoint 414. In some examples, the spiking area 410 may include an area under the sensor measurement curve 402 and above a pre-spike measurement 314. Subsequent to the endpoint 414, the sensor measurement curve 402 includes a non-spike area 320. In some examples, the non-spike area 320 may include an area under the sensor measurement curve 402 and above normal threshold 416. As illustrated by the operational example 400, a sensor measurement curve 402 for a sub-time period 308 may define a plurality of change points, data spikes, spiking areas, non-spiking areas, and/or the like. These characteristics may be leveraged by a predictive model to generate predictive insights for a sub-time period 308.

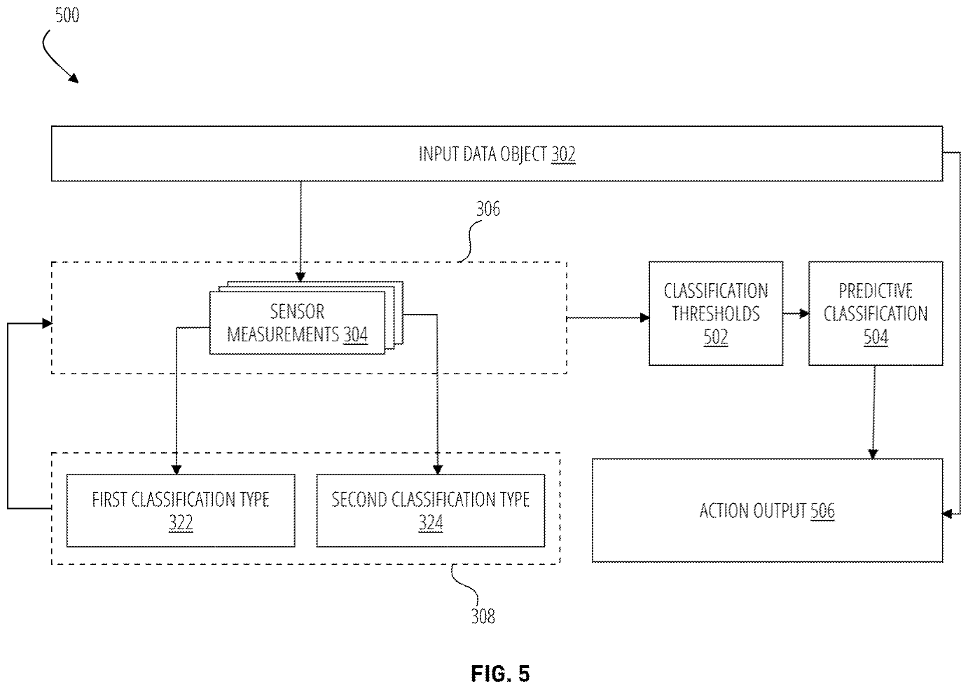

In some embodiments, the predictive insights for a sub-time period 308 may be aggregated, using one or more iterative predictive modeling techniques, across a plurality of sub-time periods for an evaluation time period to generate a predictive classification that is tailored to an input data object at a particular moment in time. An example of some of the iterative predictive modeling techniques of the present disclosure will now further be described with reference to FIG. 5.

FIG. 5 is a dataflow diagram 500 showing an iterative predictive modeling technique in accordance with some embodiments discussed herein. The dataflow diagram 500 depicts a set of data structures for determining a predictive classification 504 for an input data object 302 and initiating the performance of an action output 506. The predictive classification 504 may be iteratively determined for an evaluation time period 306 based on a plurality of classifications, such as the first classification type 322 and/or the second classification type 324, determined across a plurality sub-time periods, such as the sub-time period 308, of the evaluation time period 306. In some examples, the predictive classification 504 may be leveraged to generate and/or initiate the performance of an action output 506 for the input data object 302.

In some embodiments, a plurality of sub-time period classifications are determined for the evaluation time period 306. The plurality of sub-time period classifications may include classifications of a first classification type 322 and/or a second classification type 324 for each of a plurality of sub-time periods of the evaluation time period 306. The sub-time period classifications may be based on the prediction domain. In some examples, in a clinical domain, the plurality of sub-time period classifications may include at least one fasting phenotype classification and/or at least one post-prandial phenotype classification for each of the plurality of sub-time periods of the evaluation time period 306.

In some embodiments, the predictive classification 504 is determined based on a comparison between the plurality of sub-time period classifications and one or more classification thresholds 502. For example, the classification thresholds 502 may include a first and second threshold. The first threshold may correspond to a first classification type 322, such as a fasting phenotype classification. The second threshold may correspond to a second classification type 324, such as a post-prandial phenotype classification. For example, in a clinical domain, a fasting phenotype classification may be associated with a first threshold and a post-prandial phenotype classification may be associated with a second threshold. In some examples, each of the plurality of sub-time period classifications may be compared to first and/or second thresholds to determine the predictive classification 504.

In some embodiments, the classification thresholds 502 are data entities that describe a threshold criteria for a predictive classification 504. The classification thresholds 502 may include a threshold number of sub-time period classifications. For example, in a clinical domain, the classification thresholds 502 may include a threshold number of first phenotype classifications (e.g., fasting phenotype classifications, etc.) and/or second phenotype classifications (e.g., post-prandial phenotype classifications, etc.) over the evaluation time period 306. By way of example, the classification thresholds 502 may identify a threshold number of fasting phenotype classifications for determining whether the input data object 302 has an elevated fasting glucose. In addition, or alternatively, the classification thresholds 502 may identify a threshold number of post-prandial phenotype classifications over the evaluation time period 306 to determine whether the input data object 302 has an elevated post-prandial glucose phenotype.

In some embodiments, the predictive classification 504 is a data entity that describes a predictive output for an input data object 302. The predictive classification 504 may include one or more predictive categories for the input data object 302. The one or more predictive categories may be based on the prediction domain. In some examples, in a clinical domain, the predictive classification 504 may include at least one of an elevated fasting glucose only category, an excessive post-prandial glucose elevation only category, and/or a mixed category of elevated fasting glucose and/or excessive post-prandial glucose elevation.

By way of example, the evaluation time period 306 may include a fourteen day time period of a plurality of sensor measurements 304. Each day of the fourteen day time period may include a sub-time period 308, such that the evaluation time period 306 includes fourteen sub-time periods.

A first classification type 322 for the sub-time period 308 may include a first phenotype classification, such as a fasting phenotype classification that identifies whether the input data object 302 exhibits a fasting glucose above a threshold for a particular day in the fourteen day time period. The classification thresholds 502 may include a first threshold that defines a threshold number of sub-time periods, such as a day, for which an input data object 302 may have a fasting glucose above a threshold measurement to be categorized as having an elevated fasting glucose. By way of example, the first threshold may define a four day threshold out of a fourteen day evaluation time period. In such a case, the predictive classification 504 for the input data object 302 may include an elevated fasting glucose only category and/or a mixed category in the event that the first classification type 322 for at least four sub-time periods of the evaluation time period 306 indicates that the input data object 302 exhibited a fasting glucose above a threshold pre-spike measurement.

A second classification type 324 for the sub-time period 308 may include a second phenotype classification, such as a post-prandial phenotype classification that identifies whether the input data object 302 exhibits glucose spiking for a particular day in the fourteen day time period. The classification thresholds 502 may include a second threshold that defines a threshold number of sub-time periods, such as a day, for which an input data object 302 may have a spiking glucose to be categorized as having an excessive post-prandial glucose elevation. By way of example, the second threshold may define a four day threshold out of a fourteen day evaluation time period. In such a case, the predictive classification 504 for the input data object 302 may include an excessive post-prandial glucose elevation only category and/or a mixed category in the event that the second classification type 324 for at least four sub-time periods of the evaluation time period 306 indicates that the input data object 302 exhibited glucose spiking.

For example, in a clinical domain, the predictive classification 504 may include one of a plurality of predictive categories. The plurality of predictive categories may include a GI category indicative of an elevated fasting glucose only prediction, a GII category indicative of an excessive post-prandial glucose elevation only prediction, and/or a G-mixed category indicative of both an elevated fasting glucose and excessive post-prandial glucose elevation prediction. The GI category may be determined in the event that (i) the first classification type 322 indicates that the input data object 302 exhibited a fasting glucose above a threshold pre-spike measurement for a threshold number of sub-time periods (e.g., four days) of the evaluation time period 306 and (ii) the second classification type 324 indicates that the input data object 302 did not exhibit glucose spiking for a threshold number of sub-time periods (e.g., four days) of the evaluation time period 306. In addition, or alternatively, the GII category may be determined in the event that (i) the first classification type 322 indicates that the input data object 302 did not exhibit a fasting glucose above a threshold pre-spike measurement for a threshold number of sub-time periods (e.g., four days) of the evaluation time period 306 and (ii) the second classification type 324 indicates that the input data object 302 did exhibit glucose spiking for a threshold number of sub-time periods (e.g., four days) of the evaluation time period 306. The G-mixed category may be determined in the event that (i) the first classification type 322 indicates that the input data object 302 exhibited a fasting glucose above a threshold pre-spike measurement for a threshold number of sub-time periods (e.g., four days) of the evaluation time period 306 and (ii) the second classification type 324 indicates that the input data object 302 exhibited glucose spiking for a threshold number of sub-time periods (e.g., four days) of the evaluation time period 306.

In some embodiments, an action output 506 is generated and/or provided for the input data object 302 based on the predictive classification 504. In some embodiments, the action output 506 is a data entity that describes a predictive classification-based output for the input data object 302. The action output 506 may include one or more computing actions, such as issuing one or more alerts or notifications, generating and/or providing one or more treatment recommendations, generating and/or rendering one or more instructions, generating and/or providing one or more computing instructions, and/or the like. In some examples, in a clinical domain, the action output 506 may include a clinical plan recommendation based on the predictive classification 504 for the input data object 302.

For example, the action output 506 may include selecting a treatment plan for the predictive classification 504. By way example, input data objects classified as a GI category may receive one or more treatments and/or categories of treatments, such as Metformin, a long-acting GLP-1 agonist (e.g., exenatide XR, liraglutide), basal (long-acting) insulin, thiazolidinediones, and/or the like. Input data objects classified as a GII category may receive one or more treatments and/or categories of treatments, such as Meglitinides, Sulfonylureas, DPP4-inhibitors, bolus (short-acting) insulin at spikes, short-acting GLP-1 agonist (e.g., exenatide, lixisenatide), alpha-glucoside inhibitors, and/or the like. Input data objects classified as a G-mixed category may receive one or more treatments and/or categories of treatments, such as a GLP-1 agonist (e.g., dulaglutide), SLGT-2 inhibitors, a combination of basal and bolus insulins, and/or the like.

In some examples, the action output 506 may include one of a plurality of potential outputs for the input data object 302 and may be tailored to the specific evaluation time period 306. In some examples, some of the techniques of the present disclosure may be repeated for a plurality of evaluation time periods subsequent to the evaluation time period 306 to continuously refine the predictive classification 504 and/or an action output 506 for the input data object 302 based on current sensor measurements.

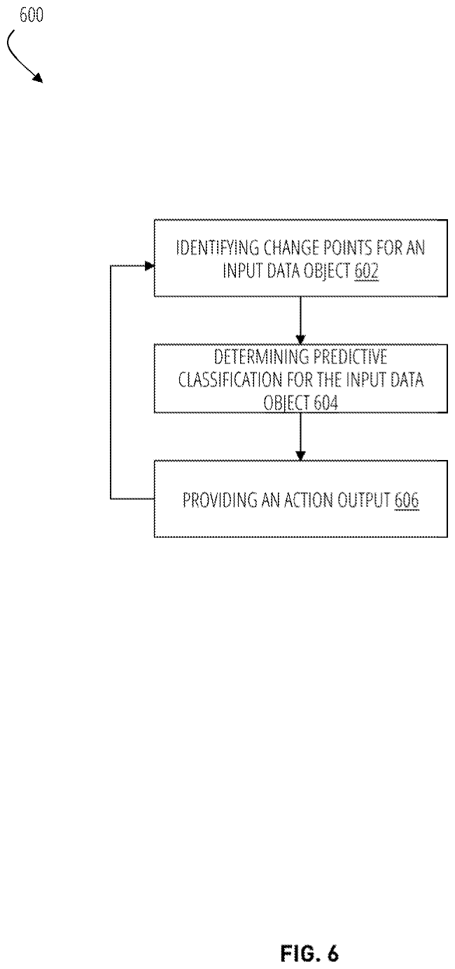

FIG. 6 is a flowchart showing an example of a process 600 for generating predictive insights for a complex prediction domain in accordance with some embodiments discussed herein. The flowchart depicts a three-stage predictive modeling technique for interpreting sensor measurements over an evaluation time period to overcome various limitations of traditional predictive modeling techniques. The three stage predictive modeling techniques may be implemented by one or more computing devices, entities, and/or systems described herein. For example, via the various steps/operations of the process 600, the computing system 100 may leverage the three-stage predictive modeling techniques to overcome the various limitations with traditional techniques by determining multiple sub-classifications of various types based on indicators in sensor measurements over an evaluation time period and leveraging the determined sub-classification to generate and provide an action output tailored to an individual input data object.

FIG. 6 illustrates an example process 600 for explanatory purposes. Although the example process 600 depicts a particular sequence of steps/operations, the sequence may be altered without departing from the scope of the present disclosure. For example, some of the steps/operations depicted may be performed in parallel or in a different sequence that does not materially impact the function of the process 600. In other examples, different components of an example device or system that implements the process 600 may perform functions at substantially the same time or in a specific sequence.

In some embodiments, the process 600 includes, at step/operation 602, identifying change points for an input data object. For example, the computing system 100 may identify, using a detection model, the one or more change points for the input data object. The input data object may include a plurality of sensor measurements that are previously generated at a predetermined timing interval over an evaluation time period. Each change point may correspond to a particular sensor measurement of the plurality of sensor measurements. In some examples, the plurality of sensor measurements may include a plurality of time-stamped continuous glucose monitoring sensor measurements.

In some examples, the detection model may include a Bayesian ensemble model. The computing system 100 may generate, using the Bayesian ensemble model, a plurality change point probabilities for the plurality of sensor measurements. The computing system 100 may identify the one or more change points based on a comparison between the plurality of change point probabilities and a sensitivity threshold. In some examples, the sensitivity threshold may be based on the input data object.

In some embodiments, the process 600 includes, at step/operation 604, determining predictive classification for the input data object. For example, the computing system 100 may determine the predictive classification for the input data object based on the change points. In some examples, the one or more sub-time period classifications may include at least one of a fasting phenotype classification and/or a post-prandial phenotype classification. For example, the predictive classification may include an identification of either or both of two type-2 diabetes subtypes including an elevated fasting glucose dominant and/or post-prandial dominant subtype.

In some examples, the computing system 100 may determine the predictive classification for the input data object based on one or more sub-time period classifications for the evaluation time period. For example, the evaluation time period may include a plurality of sub-time periods and the computing system 100 may determine a plurality of sub-time period classifications for the evaluation time period based on the sub-time periods.

In some examples, the computing system 100 may determine at least one data spike from the plurality of sensor measurements based on the one or more change points. The at least one data spike may correspond to a sub-time period of the evaluation time period. In some examples, the computing system 100 may identify at least one spiking measurement corresponding to a particular change point of the one or more change points based on a threshold measurement change. For example, the at least one spiking measurement may correspond to a sensor measurement within a measurement time period subsequent to the particular change point. The computing system 100 may determine the at least one data spike based on the at least one spiking measurement. The computing system 100 may determine one or more sub-time period classifications for the sub-time period based on the at least one data spike.

In some embodiments, the plurality of sub-time period classifications includes at least one of the fasting phenotype classification or the post-prandial phenotype classification for each of the plurality of sub-time periods. In some examples, the computing system 100 may determine the predictive classification based on a comparison between the plurality of sub-time period classifications and one or more classification thresholds. For example, the fasting phenotype classification may be associated with a first threshold and/or the post-prandial phenotype classification may be associated with a second threshold.

In some embodiments, the process 600 includes, at step/operation 606, providing an action output. For example, the computing system 100 may provide the action output 506 for the input data object based on the predictive classification. The action output may include a clinical plan recommendation. For instance, the computing system 100 may select a treatment for type-2 diabetes based on the predictive classification.

In some embodiments, the step/operations of the process 600 may include three stages of a repeatable process for an input data object. For instance, each of the stages may be repeatedly performed for each of a plurality of subsequent evaluation time periods. In this way, the action outputs for the input data object may adapt to previous action outputs and/or changes to trends of sensor measurements for an input data object over extended time periods. In this manner, the three-stage predictive modeling technique may continuously and automatically refine action outputs to accommodate for dynamic changes in data. In this manner, the three-stage predictive modeling technique of the present disclosure may be practically applied to improve upon traditional predictive modeling techniques that are (i) limited to static, group-based action outputs, (ii) fail to adapt to real-time changes in data, (iii) rely on harmful trial and error sequences, and (iv) lack precision and personalization for an individual input data object.

As discussed herein, some techniques of the present disclosure enable the generation of action outputs that may be performed to initiate one or more predictive actions to achieve real-world effects. The predictive modeling techniques of the present disclosure may be used, applied, and/or otherwise leveraged to generate predictions. These predictions may be leveraged to initiate the performance of various computing tasks that improve the performance of a computing system (e.g., a computer itself, etc.) with respect to various predictive actions performed by the computing system.

In some examples, the computing tasks may include predictive actions that may be based on a prediction domain. A prediction domain may include any environment in which computing systems may be applied to achieve real-word insights, such as predictions, and initiate the performance of computing tasks, such as predictive actions, to act on the real-world insights. These predictive actions may cause real-world changes, for example, by controlling a hardware component, providing targeted alerts, automatically allocating computing or human resources, and/or the like.

Examples of prediction domains may include financial systems, clinical systems, autonomous systems, robotic systems, and/or the like. Predictive actions in such domains may include the initiation of automated instructions across and between devices, automated notifications, automated scheduling operations, automated precautionary actions, automated security actions, automated data processing actions, automated server load balancing actions, automated computing resource allocation actions, automated adjustments to computing and/or human resource management, and/or the like.

As one example, a prediction domain may include a clinical prediction domain. In such a case, the predictive actions may include automated physician notification actions, automated patient notification actions, automated appointment scheduling actions, automated prescription recommendation actions, automated drug prescription generation actions, automated implementation of precautionary actions, automated record updating actions, automated datastore updating actions, automated hospital preparation actions, automated workforce management operational management actions, automated server load balancing actions, automated resource allocation actions, automated call center preparation actions, automated hospital preparation actions, automated pricing actions, automated plan update actions, automated alert generation actions, and/or the like.

In some embodiments, the three-stage predictive modeling techniques of process 600 are applied to initiate the performance of one or more predictive actions. As described herein, the predictive actions may depend on the prediction domain. In some examples, the computing system 100 may leverage the three-stage predictive modeling techniques to generate a predictive classification. Using the predictive classification, the computing system 100 may generate an action output that is personalized and tailored to an input data object at a particular moment in time. These predictive insights may be leveraged to initiate the performance of the one or more predictive actions within a respective prediction domain. By way of example, the prediction domain may include a clinical prediction domain and the one or more predictive actions may include performing a resource-based action (e.g., allocation of resource), generating a diagnostic report, generating action scripts, generating alerts or messages, generating one or more electronic communications, and/or the like. The one or more predictive actions may further include displaying visual renderings of the aforementioned examples of predictive actions in addition to values, charts, and representations associated with the third-party data sources and/or third-party datasets thereof.

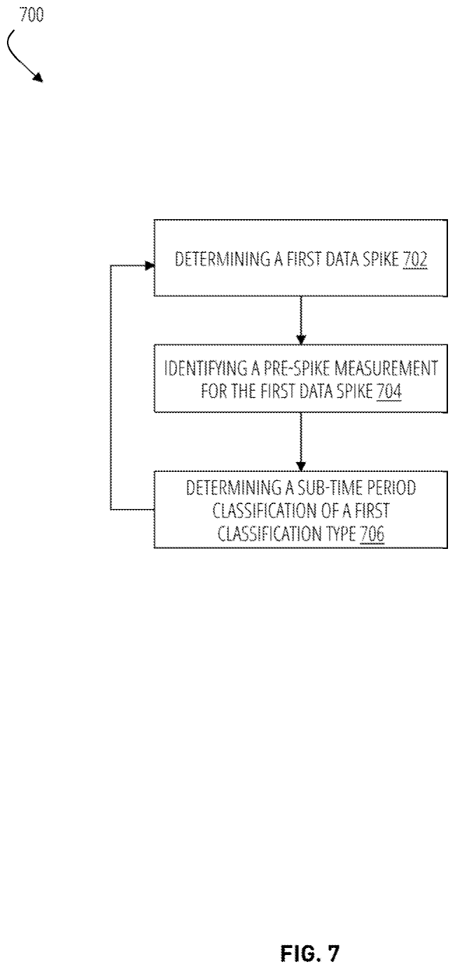

FIG. 7 is a flowchart showing an example of a process 700 for determining a first sub-time period classification for a sub-time period in accordance with some embodiments discussed herein. The flowchart depicts predictive classification techniques for generating predictive classifications from a plurality of sensor measurements to overcome various limitations of traditional predictive modeling techniques. The predictive classification techniques may be implemented by one or more computing devices, entities, and/or systems described herein. For example, via the various steps/operations of the process 700, the computing system 100 may leverage the predictive classification techniques to overcome the various limitations with traditional predictive modeling techniques by deriving previously unavailable insights from a plurality of plurality of sensor measurements.

FIG. 7 illustrates an example process 700 for explanatory purposes. Although the example process 700 depicts a particular sequence of steps/operations, the sequence may be altered without departing from the scope of the present disclosure. For example, some of the steps/operations depicted may be performed in parallel or in a different sequence that does not materially impact the function of the process 700. In other examples, different components of an example device or system that implements the process 700 may perform functions at substantially the same time or in a specific sequence.

In some embodiments, the process 700 includes, at step/operation 702, determining a first data spike. For example, the computing system 100 may determine that at least one data spike from one or more of data spikes determined for an input data object is the first data spike of a sub-time period.

In some embodiments, the process 700 includes, at step/operation 704, identifying a pre-spike measurement for the first data spike. For example, the computing system 100 may identify a pre-spike measurement for the at least one data spike identified as the first data spike. For instance, the computing system 100 may identify the pre-spike measurement in response to determining that the at least one data spike is the first data spike. The pre-spike measurement may be a sensor measurement of the plurality of sensor measurements for the input data object that corresponds to a pre-spike measurement time period preceding a start time of the at least one data spike.

In some embodiments, the process 700 includes, at step/operation 706, determining a sub-time period classification of a first classification type for the sub-time period. For example, the computing system 100 may determine the sub-time period classification for the sub-time period. In some examples, the sub-time period classification may be a fasting phenotype classification. For instance, the computing system 100 may determine the fasting phenotype classification for the sub-time period based on a comparison between the pre-spike measurement and a threshold pre-spike measurement.

In some examples, the threshold pre-spike measurement may correspond to a previous evaluation time period for the input data object. For example, the sub-time period classification may be determined for each sub-time period of an evaluation time period. The computing system 100 may leverage the plurality of sub-time period classifications to derive one or more predictive insights for an input data object. These insights may be leveraged to generate and provide an action output for an input data object that is tailored and personalized to a particular evaluation time period.

Using the predictive classification techniques of the process 700, additional sub-time period classifications may be determined for any number of sub-time periods in any number of evaluation time periods. At each subsequent evaluation time period, the threshold pre-spike measurement for determining sub-time period classifications may be refined to tailor the sub-time period classifications to historical observations and/or real-time data trends for the input data object. In this manner, the predictive classification techniques of the process 700 may be practically applied to improve upon traditional predictive modeling techniques that are (i) limited to static, group-based action outputs, (ii) fail to adapt to real-time changes in data, (iii) rely on harmful trial and error sequences, and (iv) lack precision and personalization for an individual input data object.

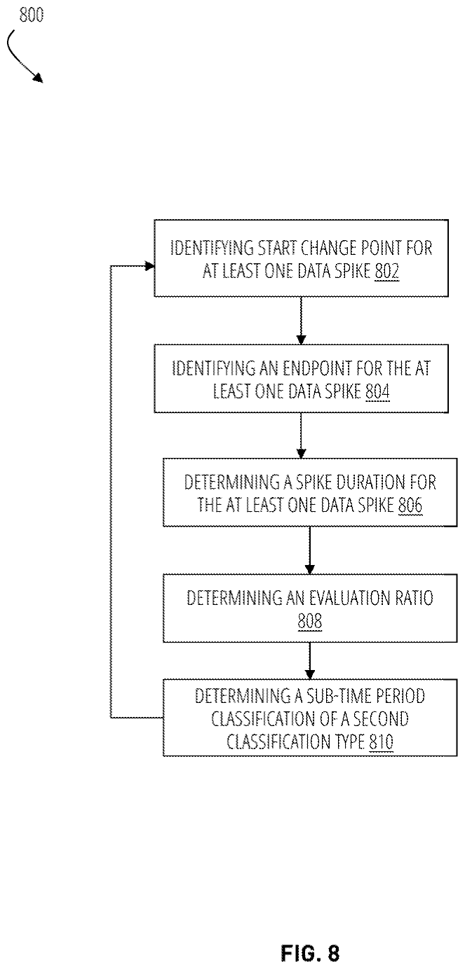

FIG. 8 is a flowchart showing an example of a process 800 for determining a second sub-time period classification for a sub-time period in accordance with some embodiments discussed herein. The flowchart depicts predictive classification techniques for generating predictive classifications from a plurality of sensor measurements to overcome various limitations of traditional predictive modeling techniques. The predictive classification techniques may be implemented by one or more computing devices, entities, and/or systems described herein. For example, via the various steps/operations of the process 700, the computing system 100 may leverage the predictive classification techniques to overcome the various limitations with traditional predictive modeling techniques by deriving previously unavailable insights from a plurality of plurality of sensor measurements.

FIG. 8 illustrates an example process 800 for explanatory purposes. Although the example process 800 depicts a particular sequence of steps/operations, the sequence may be altered without departing from the scope of the present disclosure. For example, some of the steps/operations depicted may be performed in parallel or in a different sequence that does not materially impact the function of the process 800. In other examples, different components of an example device or system that implements the process 800 may perform functions at substantially the same time or in a specific sequence.

In some embodiments, the process 800 includes, at step/operation 802, identifying a start change point for at least one data spike. For example, the computing system 100 may identify the start change point for the at least one data spike. The start change point may correspond to a first sensor measurement.

In some embodiments, the process 800 includes, at step/operation 804, identifying an endpoint for the at least one data spike. For example, the computing system 100 may identify the endpoint for the at least one data spike. In some examples, the endpoint may correspond to a post-spike sensor measurement that is subsequent to the start change point and is within a threshold margin of the first sensor measurement.

In some embodiments, the process 800 includes, at step/operation 806, determining a spike duration for the at least one data spike. For example, the computing system 100 may determine the spike duration for the at least one data spike based on the start change point and the endpoint.

In some embodiments, the process 800 includes, at step/operation 808, determining an evaluation ratio. For example, the computing system 100 may determine the evaluation ratio based on the spike duration. The evaluation ratio may be indicative of a first measurement area corresponding to the at least one data spike relative to a second measurement area corresponding to one or more portions of the sub-time period that are outside the at least one data spike.

In some embodiments, the process 800 includes, at step/operation 810, determining a sub-time period classification of a second classification type. For example, the computing system 100 may determine the sub-time period classification of a second classification type. In some examples, the sub-time period classification may include a post-prandial phenotype classification. The computing system 100 may determine the post-prandial phenotype classification for the sub-time period based on a comparison between the evaluation ratio and a threshold ratio.

In some examples, the threshold ratio may correspond to a previous evaluation time period for the input data object. For example, the sub-time period classification may be determined for each sub-time period of an evaluation time period. The computing system 100 may leverage to plurality of sub-time period classifications to derive one or more predictive insights for an input data object. These insights may be leveraged to generate and provide an action output for an input data object that is tailored and personalized to a particular evaluation time period.

Using the predictive classification techniques of the process 800, additional sub-time period classifications may be determined for any number of sub-time periods in any number of evaluation time periods. At each subsequent evaluation time period, the threshold ratio for determining the sub-time period classification may be refined to tailor the sub-time period classifications to historical observations and/or real-time data trends for the input data object. In this manner, the predictive classification techniques of the process 800 may be practically applied to improve upon traditional predictive modeling techniques that are (i) limited to static, group-based action outputs, (ii) fail to adapt to real-time changes in data, (iii) rely on harmful trial and error sequences, and (iv) lack precision and personalization for an individual input data object.

### VI. CONCLUSION

Many modifications and other embodiments will come to mind to one skilled in the art to which the present disclosure pertains having the benefit of the teachings presented in the foregoing descriptions and the associated drawings. Therefore, it is to be understood that the present disclosure is not to be limited to the specific embodiments disclosed and that modifications and other embodiments are intended to be included within the scope of the appended claims. Although specific terms are employed herein, they are used in a generic and descriptive sense only and not for purposes of limitation.

### VII. EXAMPLES

Example 1. A computer-implemented method comprising: identifying, by one or more processors and using a detection model, one or more change points for an input data object, wherein (a) the input data object comprises a plurality of sensor measurements, (b) each sensor measurement of the plurality of sensor measurements was previously generated at a predetermined timing interval over an evaluation time period, and (c) each change point corresponds to a particular sensor measurement of the plurality of sensor measurements; determining, by the one or more processors, at least one data spike from the plurality of sensor measurements based on the one or more change points, wherein the at least one data spike corresponds to a sub-time period of the evaluation time period; determining, by the one or more processors, one or more sub-time period classifications based on the at least one data spike; determining, by the one or more processors, a predictive classification for the input data object based on the one or more sub-time period classifications; and providing, by the one or more processors, an action output for the input data object based on the predictive classification.

Example 2. The computer-implemented method of example 1, wherein the plurality of sensor measurements comprises a plurality of time-stamped continuous glucose monitoring sensor measurements.

Example 3. The computer-implemented method of any of the preceding examples, wherein determining the at least one data spike comprises: identifying at least one spiking measurement corresponding to a particular change point of the one or more change points based on a threshold measurement change, wherein the at least one spiking measurement corresponds to a sensor measurement within a measurement time period subsequent to the particular change point; and determining the at least one data spike based on the at least one spiking measurement.

Example 4. The computer-implemented method of any of the preceding examples, wherein the detection model comprises a Bayesian ensemble model, and wherein identifying the one or more change points comprises: generating, using the Bayesian ensemble model, a plurality change point probabilities for the plurality of sensor measurements; and identifying the one or more change points based on a comparison between the plurality of change point probabilities and a sensitivity threshold.

Example 5. The computer-implemented method of example 4, wherein the sensitivity threshold is based on the input data object.

Example 6. The computer-implemented method of any of the preceding examples, wherein the one or more sub-time period classifications comprise at least one of a fasting phenotype classification or a post-prandial phenotype classification.

Example 7. The computer-implemented method of example 6, wherein determining one or more sub-time period classifications based on the at least one data spike comprise: determining that the at least one data spike is a first data spike for the sub-time period; in response to determining that the at least one data spike is the first data spike: identifying a pre-spike measurement for the at least one data spike, wherein the pre-spike measurement is a sensor measurement of the plurality of sensor measurements that corresponds to a pre-spike measurement time period preceding a start time of the at least one data spike; and determining the fasting phenotype classification for the sub-time period based on a comparison between the pre-spike measurement and a threshold pre-spike measurement.

Example 8. The computer-implemented method of example 7, wherein the threshold pre-spike measurement corresponds to a previous evaluation time period for the input data object.

Example 9. The computer-implemented method of any of examples 6 through 8, wherein determining one or more sub-time period classifications based on the at least one data spike comprise: identifying a start change point for the at least one data spike, wherein the start change point corresponds to a first sensor measurement; identifying an endpoint for the at least one data spike, wherein the endpoint corresponds to a post-spike sensor measurement that is subsequent to the start change point and is within a threshold margin of the first sensor measurement; determining a spike duration for the at least one data spike based on the start change point and the endpoint; determining an evaluation ratio based on the spike duration, wherein the evaluation ratio is indicative of a first measurement area corresponding to the at least one data spike relative to a second measurement area corresponding to one or more portions of the sub-time period that are outside the at least one data spike; and determining the post-prandial phenotype classification for the sub-time period based on a comparison between the evaluation ratio and a threshold ratio.

Example 10. The computer-implemented method of example 9, wherein the threshold ratio corresponds to a previous evaluation time period for the input data object.

Example 11. The computer-implemented method any of examples 6 through 10, wherein the evaluation time period comprises a plurality of sub-time periods, and wherein determining the predictive classification comprises: determining a plurality of sub-time period classifications for the evaluation time period, wherein the plurality of sub-time period classifications comprise at least one of the fasting phenotype classification or the post-prandial phenotype classification for each of the plurality of sub-time periods; and determining the predictive classification based on a comparison between the plurality of sub-time period classifications and one or more classification thresholds.

Example 12. The computer-implemented method of example 11, wherein the fasting phenotype classification is associated with a first threshold, and the post-prandial phenotype classification is associated with a second threshold.

Example 13. The computer-implemented method of any of the preceding examples, wherein the action output comprises a clinical plan recommendation.

Example 14. A computing apparatus comprising memory and one or more processors communicatively coupled to the memory, the one or more processors configured to: identify, using a detection model, one or more change points for an input data object, wherein (a) the input data object comprises a plurality of sensor measurements, (b) each sensor measurement of the plurality of sensor measurements was previously generated at a predetermined timing interval over an evaluation time period, and (c) each change point corresponds to a particular sensor measurement of the plurality of sensor measurements; determine at least one data spike from the plurality of sensor measurements based on the one or more change points, wherein the at least one data spike corresponds to a sub-time period of the evaluation time period; determine one or more sub-time period classifications based on the at least one data spike; determine a predictive classification for the input data object based on the one or more sub-time period classifications; and provide an action output for the input data object based on the predictive classification.

Example 15. The computing apparatus of example 14, wherein the plurality of sensor measurements comprises a plurality of time-stamped continuous glucose monitoring sensor measurements.

Example 16. The computing apparatus of examples 14 or 15, wherein determining the at least one data spike comprises: identifying at least one spiking measurement corresponding to a particular change point of the one or more change points based on a threshold measurement change, wherein the at least one spiking measurement corresponds to a sensor measurement within a measurement time period subsequent to the particular change point; and determining the at least one data spike based on the at least one spiking measurement.

Example 17. The computing apparatus of any of examples 14 through 16, wherein the detection model comprises a Bayesian ensemble model, and wherein identifying the one or more change points comprises: generating, using the Bayesian ensemble model, a plurality change point probabilities for the plurality of sensor measurements; and identifying the one or more change points based on a comparison between the plurality of change point probabilities and a sensitivity threshold.

Example 18. The computing apparatus of example 17, wherein the sensitivity threshold is based on the input data object.

Example 19. One or more non-transitory computer-readable storage media including instructions that, when executed by one or more processors, cause the one or more processors to: identify, using a detection model, one or more change points for an input data object, wherein (a) the input data object comprises a plurality of sensor measurements, (b) each sensor measurement of the plurality of sensor measurements was that are previously generated at a predetermined timing interval over an evaluation time period, and (c) each change point corresponds to a particular sensor measurement of the plurality of sensor measurements; determine at least one data spike from the plurality of sensor measurements based on the one or more change points, wherein the at least one data spike corresponds to a sub-time period of the evaluation time period; determine one or more sub-time period classifications based on the at least one data spike; determine a predictive classification for the input data object based on the one or more sub-time period classifications; and provide an action output for the input data object based on the predictive classification.

Example 20. The one or more non-transitory computer-readable storage media of example 19, wherein the one or more sub-time period classifications comprise at least one of a fasting phenotype classification or a post-prandial phenotype classification.

1. A computer-implemented method comprising:
identifying, by one or more processors and using a detection model, one or more change points for an input data object, wherein (a) the input data object comprises a plurality of sensor measurements, (b) each sensor measurement of the plurality of sensor measurements was previously generated at a predetermined timing interval over an evaluation time period, and (c) each change point corresponds to a particular sensor measurement of the plurality of sensor measurements;
determining, by the one or more processors, at least one data spike from the plurality of sensor measurements based on the one or more change points, wherein the at least one data spike corresponds to a sub-time period of the evaluation time period;
determining, by the one or more processors, one or more sub-time period classifications based on the at least one data spike;
determining, by the one or more processors, a predictive classification for the input data object based on the one or more sub-time period classifications; and
providing, by the one or more processors, an action output for the input data object based on the predictive classification.

2. The computer-implemented method of claim 1, wherein the plurality of sensor measurements comprises a plurality of time-stamped continuous glucose monitoring sensor measurements.

3. The computer-implemented method of claim 1, wherein determining the at least one data spike comprises:
identifying at least one spiking measurement corresponding to a particular change point of the one or more change points based on a threshold measurement change, wherein the at least one spiking measurement corresponds to a sensor measurement within a measurement time period subsequent to the particular change point; and
determining the at least one data spike based on the at least one spiking measurement.

4. The computer-implemented method of claim 1, wherein the detection model comprises a Bayesian ensemble model, and wherein identifying the one or more change points comprises:
generating, using the Bayesian ensemble model, a plurality change point probabilities for the plurality of sensor measurements; and
identifying the one or more change points based on a comparison between the plurality of change point probabilities and a sensitivity threshold.

5. The computer-implemented method of claim 4, wherein the sensitivity threshold is based on the input data object.

6. The computer-implemented method of claim 1, wherein the one or more sub-time period classifications comprise at least one of a fasting phenotype classification or a post-prandial phenotype classification.

7. The computer-implemented method of claim 6, wherein determining one or more sub-time period classifications based on the at least one data spike comprise:
determining that the at least one data spike is a first data spike for the sub-time period;
in response to determining that the at least one data spike is the first data spike:
identifying a pre-spike measurement for the at least one data spike, wherein the pre-spike measurement is a sensor measurement of the plurality of sensor measurements that corresponds to a pre-spike measurement time period preceding a start time of the at least one data spike; and
determining the fasting phenotype classification for the sub-time period based on a comparison between the pre-spike measurement and a threshold pre-spike measurement.

8. The computer-implemented method of claim 7, wherein the threshold pre-spike measurement corresponds to a previous evaluation time period for the input data object.

9. The computer-implemented method of claim 6, wherein determining one or more sub-time period classifications based on the at least one data spike comprise:
identifying a start change point for the at least one data spike, wherein the start change point corresponds to a first sensor measurement;
identifying an endpoint for the at least one data spike, wherein the endpoint corresponds to a post-spike sensor measurement that is subsequent to the start change point and is within a threshold margin of the first sensor measurement;
determining a spike duration for the at least one data spike based on the start change point and the endpoint;
determining an evaluation ratio based on the spike duration, wherein the evaluation ratio is indicative of a first measurement area corresponding to the at least one data spike relative to a second measurement area corresponding to one or more portions of the sub-time period that are outside the at least one data spike; and
determining the post-prandial phenotype classification for the sub-time period based on a comparison between the evaluation ratio and a threshold ratio.

10. The computer-implemented method of claim 9, wherein the threshold ratio corresponds to a previous evaluation time period for the input data object.

11. The computer-implemented method of claim 6, wherein the evaluation time period comprises a plurality of sub-time periods, and wherein determining the predictive classification comprises:
determining a plurality of sub-time period classifications for the evaluation time period, wherein the plurality of sub-time period classifications comprise at least one of the fasting phenotype classification or the post-prandial phenotype classification for each of the plurality of sub-time periods; and
determining the predictive classification based on a comparison between the plurality of sub-time period classifications and one or more classification thresholds.

12. The computer-implemented method of claim 11, wherein the fasting phenotype classification is associated with a first threshold, and the post-prandial phenotype classification is associated with a second threshold.

13. The computer-implemented method of claim 1, wherein the action output comprises a clinical plan recommendation.

14. A computing apparatus comprising memory and one or more processors communicatively coupled to the memory, the one or more processors configured to:
identify, using a detection model, one or more change points for an input data object, wherein (a) the input data object comprises a plurality of sensor measurements, (b) each sensor measurement of the plurality of sensor measurements was previously generated at a predetermined timing interval over an evaluation time period, and (c) each change point corresponds to a particular sensor measurement of the plurality of sensor measurements;
determine at least one data spike from the plurality of sensor measurements based on the one or more change points, wherein the at least one data spike corresponds to a sub-time period of the evaluation time period;
determine one or more sub-time period classifications based on the at least one data spike;
determine a predictive classification for the input data object based on the one or more sub-time period classifications; and
provide an action output for the input data object based on the predictive classification.

15. The computing apparatus of claim 14, wherein the plurality of sensor measurements comprises a plurality of time-stamped continuous glucose monitoring sensor measurements.

16. The computing apparatus of claim 14, wherein determining the at least one data spike comprises:
identifying at least one spiking measurement corresponding to a particular change point of the one or more change points based on a threshold measurement change, wherein the at least one spiking measurement corresponds to a sensor measurement within a measurement time period subsequent to the particular change point; and
determining the at least one data spike based on the at least one spiking measurement.

17. The computing apparatus of claim 14, wherein the detection model comprises a Bayesian ensemble model, and wherein identifying the one or more change points comprises:
generating, using the Bayesian ensemble model, a plurality change point probabilities for the plurality of sensor measurements; and
identifying the one or more change points based on a comparison between the plurality of change point probabilities and a sensitivity threshold.

18. The computing apparatus of claim 17, wherein the sensitivity threshold is based on the input data object.

19. One or more non-transitory computer-readable storage media including instructions that, when executed by one or more processors, cause the one or more processors to:
identify, using a detection model, one or more change points for an input data object, wherein (a) the input data object comprises a plurality of sensor measurements, (b) each sensor measurement of the plurality of sensor measurements was that are previously generated at a predetermined timing interval over an evaluation time period, and (c) each change point corresponds to a particular sensor measurement of the plurality of sensor measurements;
determine at least one data spike from the plurality of sensor measurements based on the one or more change points, wherein the at least one data spike corresponds to a sub-time period of the evaluation time period;
determine one or more sub-time period classifications based on the at least one data spike;
determine a predictive classification for the input data object based on the one or more sub-time period classifications; and
provide an action output for the input data object based on the predictive classification.

20. The one or more non-transitory computer-readable storage media of claim 19, wherein the one or more sub-time period classifications comprise at least one of a fasting phenotype classification or a post-prandial phenotype classification.
